(19) United States
(12) Patent Application Publication
(10) Pub. No.: US 2009/0326196 A1
(43) Pub. Date: Dec. 31, 2009

(54) Proteins Containing a Fluorinated Amino Acid, and Methods of Using Same

(75) Inventors: Krishna Kumar, Cambridge, MA (US); He Meng, Newtonville, MA (US)

Correspondence Address: FOLEY HOAG, LLP;PATENT GROUP, WORLD TRADE CENTER WEST, 155 SEAPORT BLVD, BOSTON, MA, 02110, US

(73) Assignee: Trustees of Tufts College

(21) Appl. No.: 12/161,251

(22) PCT Filed: Jan. 17, 2007
(86) PCT No.: PCT/US07/01184

§ 371 Date: Nov. 18, 2008

Related U.S. Application Data
(60) Provisional application No. 60/759,441, filed on Jan. 17, 2006.

(51) Int. Cl. C07K 14/00; C07K 7/00

(52) U.S. Cl. 530/324; 530/326; 530/328

(57) ABSTRACT

One aspect of the invention relates to a polypeptide comprising at least one fluorinated amino acid. Another aspect of the invention relates to a method for modifying a first polypeptide, comprising replacing at least one amino acid in said first polypeptide with a fluorinated amino acid, thereby producing a second polypeptide with increased stability relative to said first polypeptide.

### RELATED APPLICATIONS

This application claims the benefit of priority to U.S. Provisional Patent Application Ser. No. 60/759,441, filed Jan. 17, 2006.

### BACKGROUND OF THE INVENTION

Proteins fold to adopt unique three dimensional structures, usually as a result of multiple non-covalent interactions that contribute to their conformational stability. Creighton, T. E. Proteins: Structures and Molecular Properties; 2nd ed.; W. H. Freeman: New York, 1993. Removal of hydrophobic surface area from aqueous solvent plays a dominant role in stabilizing protein structures. Tanford, C. Science 1978, 200, 1012-1018; and Kauzmann, W. Adv. Protein Chem. 1959, 14, 1-63. For instance, a buried leucine or phenylalanine residue can contribute ˜2-5 kcal/mol in stability when compared to alanine. Although hydrogen bonds and salt bridges, when present in hydrophobic environments, can contribute as much as 3 kcal/mol to protein stability, solvent exposed electrostatic interactions contribute far less, usually ≦0.5 kcal/mol. Yu, Y. et al. J. Mol. Biol. 1996, 255, 367-372; and Lumb, K. J.; Kim, P. S. Science 1995, 268, 436-439. Hydrogen bonds between small polar side chains and backbone amides can be worth 1-2 kcal/mol, as seen in the case of N-terminal helical caps. Aurora, R.; Rose, G. D. Protein Sci. 1998, 7, 21-38. The energetic balance of these intramolecular forces and interactions with the solvent determines the shape and the stability of the fold.

While electrostatic interactions in designed structures can provide conformational specificity at the expense of thermodynamic stability, hydrophobic interactions afford a very powerful driving force for stabilizing structures. Recent studies have focused on the introduction of non-proteinogenic, fluorine-containing amino acids as a means for increasing hydrophobicity without significant concurrent alteration of protein structure. Bilgiçer, B.; Fichera, A.; Kumar, K. J. Am. Chem. Soc. 2001, 123, 4393-4399; and Tang, Y. et al. Biochemistry 2001, 40, 2790-2796. The estimated average volumes of CH₂ and CH₃ groups are 27 and 54 Å³, respectively, as compared to the much larger 38 and 92 Å³ for CF₂ and CF₃ groups. Israelachvili, J. N. et al. Biochim. Biophysica Acta 1977, 470, 185-201. Given that the hydrophobic effect is roughly proportional to the solvent exposed surface area, the large size and volume of trifluoromethyl groups, in combination with the low polarizability of fluorine atoms, results in enhanced hydrophobicity. Tanford, C. The Hydrophobic Effect: Formation of Micelles and Biological Membranes; 2d ed.; Wiley: New York, 1980. Indeed, partition coefficients point to the superior hydrophobicity of CF₃ (Π=1.07) over CH₃ (Π=0.50) groups. Resnati, G. Tetrahedron 1993, 49, 9385-9445. The low polarizability of fluorine also results in low cohesive energy densities of liquid fluorocarbons and is manifested in their low propensities for intermolecular interactions. Riess, J. G. Colloid Surf:-A 1994, 84,33-48; and Scott, R. L. J. Am. Chem. Soc. 1948, 70, 4090-4093. These unique properties of fluorine simultaneously bestow hydrophobic and lipophobic character to biopolymers with high fluorine content. Marsh, E. N. G. Chem. Biol. 2000, 7, R153-R157.

Introduction of amino acids containing terminal trifluoromethyl groups at appropriate positions on protein folds increases the thermal stability and enhances resistance to chemical denaturants. Bilgiçer, B.; Fichera, A.; Kumar, K. J. Am. Chem. Soc. 2001, 123, 4393-4399; Tang, Y. et al. Biochemistry 2001, 40, 2790-2796. Furthermore, specific protein-protein interactions can be programmed by the use of fluorocarbon and hydrocarbon side chains. Bilgiçer, B.; Xing, X.; Kumar, K. J. Am. Chem. Soc. 2001, 123, 11815-11816. Because specificity is determined by the thermodynamic stability of all possible protein-protein interactions, a detailed -fundamental understanding of the various combinations is essential.

The so-called “leucine zipper” protein motif, originally discovered in DNA-binding proteins but also found in protein-binding proteins, consists of a set of four or five consecutive leucine residues repeated every seven amino acids in the primary sequence of a protein. In a helical configuration, a protein containing a leucine zipper motif presents a line of leucines on one side of the helix. With two such helixes alongside each other, the arrays of leucines can interdigitate like a zipper and/or form side-to-side contacts, thus forming a stable link between the two helices. Moreover, an increase in the hydrophobicity of the leucine sidechains, e.g., by substitution of hydrogens with fluorines, in a leucine zipper motif should increase the strength of the zipper.

Selective fluorination of biologically active compounds is often accompanied by dramatic changes in physiological activities. Welch, T.; Eswarakrishnan, S. Fluorine in Bioorganic Chemistry; Wiley-Interscience: New York, 1991 and references cited therein; Fluorine-containing Amino Acids; Kukhar, V. P., Soloshonok, V. A., Eds.; John Wiley & Sons: Chichester, 1994; Williams, R. M. Synthesis of Optically Active α-Amino Acids, Pergamon Press: Oxford, 1989; Ojima, I. et al. J. Org. Chem. 1989, 54, 4511-4522; Tsushima, T. et al. Tetrahedron 1988, 44, 5375-5387; Weinges, K.; Kromm, E. Liebigs Ann. Chem. 1985,90-102; Eberle, M. K. et al. Helv. Chim. Acta 1998, 81, 182-186; Tolman, V. Amino Acids 1996, 11, 15-36. Further, fluorinated amino acids have been synthesized and studied as potential inhibitors of enzymes and as therapeutic agents. Kollonitsch, J. et al. Nature 1978, 274, 906-908. Trifluoromethyl containing amino acids acting as potential antimetabolites have also been reported. Walborsky, H. M.; Baum, M. E. J. Am. Chem. Soc. 1958, 80, 187-192; Walborsky, H. M. et al. J. Am. Chem. Soc. 1955, 77, 3637-3640; Hill, H. M. et al. J. Am. Chem. Soc. 1950, 72, 3289-3289.

The emergence of bacterial resistance to common antibiotics poses a serious threat to human health and has rekindled interest in antimicrobial peptides. Both plants and animals have an arsenal of short peptides that are diverse in structure and are deployed against microbial pathogens. The common distinguishing characteristic among these peptides is their ability to form facially amphipathic conformations, segregating cationic and hydrophobic side chains. Both α-helical (magainins and cecropins) and β-sheet (bactenecins and defensins) secondary structure elements are represented. Most eukaryotes express a combination of such peptides from many different classes within tissues that provide the first line of defense against invading microbes. Coates, A. et al. Nat. Rev. Drug Discov. 2002, 1, 895-910; Zasloff, M. Nature 2002, 415, 389-395; Tossi, A. et al. Biopolymers 2000, 55, 4-30; Ganz, T. Nat. Rev. Immunol. 2003, 3, 710-720. The architectural details reveal the mechanism of action—positive charges help the peptides seek out negatively charged bacterial membranes and the interaction of the hydrophobic side chains with the acyl chain region of lipid bilayers eventually leads to membrane rupture. As a result of the broad spectrum activity and ancient lineage of these peptides, it has been suggested that bacterial resistance may be completely thwarted or slowed down enough to offer a long therapeutic lifetime for suitable candidates. Brogden, K. A. Nat. Rev. Microbiol. 2005, 3, 238-250; Hilpert, K. et al. Nat. Biotechnol. 2005, 23, 1008-1012.

Strategies to modulate antimicrobial activity of host defense peptides have relied mainly on substitution at single (or multiple) sites by one of the other nineteen natural amino acids. This approach has resulted in several improved variants, most notably the [Ala] magainin II amide. Fernandez-Lopez, S. et al. Nature 2001, 412,452-455; Tang, Y. et al. Biochemistry 2001, 40,2790-2796; Kobayashi, S. et al. Biochemistry 2004, 43, 15610-15616. On the other hand, general principles gleaned from the study of natural peptides have been utilized in the design of antimicrobial peptides and polymers using non-natural building blocks. Several of these constructs based on β-peptides, D,L-α-peptides and arylamide polymers show impressive bactericidal activity. Zasloff, M. Proc. Natl. Acad. Sci. U.S.A. 1987, 84, 5449-5453; Chen, H. C. et al. FEBS Lett. 1988, 236, 462-466; Porter, E. A. et al. Nature 2000, 404, 565-565; Porter, E. A. et al. J. Am. Chem. Soc. 2002, 124, 7324-7330; Schmitt, M. A. et al. J. Am. Chem. Soc. 2004, 126, 6848-6849; Fernandez-Lopez, S. et al. Nature 2001, 412, 452-455; Tew, G. N. et al. Proc. Natl. Acad. Sci. U.S.A. 2002, 99, 5110-5114.

The mammalian hormone Glucagon-like peptide 1 (7-36) amide (GLP-1) has great potential as an antidiabetic agent. Meier, J. J.; Nauck, M. A. Diabetes-Metab. Res. Rev. 2005, 21, 91. GLP-1 binds to the GLP-1R on the pancreatic β cells and the hydrophobic interactions are likely the major driving force responsible for the association of this amphiphilic α-helical peptide to its receptor. Wilmen, A. et al. Peptides 1997, 18, 301; Adelhorst, K. et al. J. Biol. Chem. 1994, 269, 6275. Along with other factors, GLP-1 is synthetically accessible, has a fast enzymatic clearance rate, and has a hydrophobic receptor binding surface. GLP-1, a 30-residue peptide secreted from intestine L cells in response to food intake, has unique insulinotropic and growth factor like properties. Upon binding to its specific seven transmembrane G protein-coupled receptor (GLP-1R) mainly through hydrophobic interaction, (1) GLP-1 potentiates glucose-dependent insulin secretion, stimulates pancreatic β-cell proliferation and neogenesis as well as suppresses apoptosis, inhibits glucagon secretion, delays gastrointestinal motility, and induces satiety. Holz, G. G. et al. Nature 1993, 361, 362; Ammala, C. et al. Nature 1993, 363, 356; Vilsboll, T.; Holst, J. J. Diabetologia 2004, 47, 357; Brubaker, P. L.; Drucker, D. J. Endocrinology 2004, 145, 2653. Unlike other antidiabetic therapeutics (e.g. sulfonylurea), no hypoglycemia was found as adverse effect with administration of GLP-1. However, the clinical utility of native GLP-1 is severely hampered by its rapid enzymatic deactivation by the serine protease dipeptidyl peptidase IV (DPP IV, EC 3.4.14.5), to deliver an antagonist or partial agonist GLP-1(9-36) amide. Small molecular agonists capable of mimic GLP-1 actions are of course highly desired, however, discovered small molecule ligands turned out to be antagonists so far. Tibaduiza, E. C.; Chen, C.; Beinborn, M. J. Biol. Chem. 2001, 276, 37787. For this reason, peptide-based agonists to GLP-1R with longer half-life time still are the major focuses in past decades, as exemplified by exendin 4, albumin-bound and lipidated GLP-1 derivatives NN211 and CJC-1131, with a prolonged half-life time in humans ranging from several hours to more than ten days. Knudsen, L. B. J. Med. Chem. 2004, 47, 4128.

### SUMMARY OF THE INVENTION

Remarkably, we have discovered that peptide assemblies that incorporate highly fluorinated residues have higher thermal and chemical stability. Furthermore, appropriately designed fluorinated peptides show higher affinity for membranes as in the case of cell lytic melittin, and can also direct discrete oligomer formation in biological membranes. Bilgiçer, B.; Fichera, A.; Kumar, K. J. Am. Chem. Soc. 2001, 123, 4393-4399; Bilgiçer, B.; Kumar, K. Proc. Natl. Acad. Sci. U.S.A. 2004, 101, 15324-15329; Bilgiçer, B. et al. J. Am. Chem. Soc. 2001, 123, 11815-11816, Niemz, A.; Tirrell, D. A. J. Am. Chem. Soc. 2001, 123, 7407-7413. We have discovered that increased membrane affinity and greater structural stability yields peptide variants that are more stable to proteases and also results in an increase in the potency of antimicrobial peptides. We describe herein inter alia the design, synthesis, characterization and enhanced thermal and chemical stability and biological activities of peptide systems comprising fluorinated amino acids.

Another aspect of the present invention relates to the enhancement of potency, enhanced thermal and chemical stability, and increased protease resistance of biologically active peptides via the incorporation of fluorinated amino acid side chains.

Another aspect of the invention relates to the fluorination effects on a hormonal peptide, GLP-1, regarding the binding affinity to its receptor, signal transduction ability, and enzymatic stability. We show that incorporation of highly fluorinated amino acids led to the enhanced enzymatic stability and preserved biological activity in terms of efficacy. These results indicate that fluorinated amino acids could be potentially useful for modifying peptide drug candidates

### BRIEF DESCRIPTION OF THE FIGURES

FIG. 1 depicts sequences of antimicrobial peptides. The numbers in parentheses are the net charge at pH 7.40 and the percentage solvent B (9:1:0.007 CH—₃CN/H₂O/CF₃CO₂H) required for elution on RP-BPLC on a J. T. Baker C18 column (5 μm, 4×250 mm), respectively.

FIG. 2(a) depicts helical wheel diagrams using a pitch of 3.6 residues per turn for the peptides and sites of fluorination: (A) buforin series; (B) magainin series; and (C) NMR structure of magainin 2 in dodecylphosphocholine micelles (PDB code: 2 mag) indicating the sites of fluorination (residues Leu 6 and Ile 20) in M2F2 and (residues Leu 6, Ala 9, Gly 13, Val 17 and Ile 20) in M2F5, shown in space-filling depiction. Residues indicated in blue in (A) and (B) were replaced with hexafluoroleucine to yield the fluorinated analogues. For the buforin series peptides, both leucine residues on the hydrophobic face were replaced by hexafluoro-leucine that form part of the putative DNA/RNA binding sequence.

FIG. 3 contains Table 1 which provides MIC and Percentage Hemolysis Values for selected peptides of the invention.

FIG. 4(A) depicts the relative rates of proteolytic cleavage of fluorinated peptides compared to controls; (B) fragment M*(1-14) appearance and degradation; and (C) fragment BII1*(6-21) appearance and degradation.

FIG. 5 depicts a method for the optical resolution of trifluoromethyl amino acids. The racemic mixture is N-acylated with acetic anhydride (90% yield), followed by enzymatic cleavage to yield the α-S isomer (99% yield). The stereochemistry at the β (trifluorovaline) and γ (trifluoroleucine) carbons is still unresolved. A method for the production of the N-t-Boc-protected amino acid is also depicted.

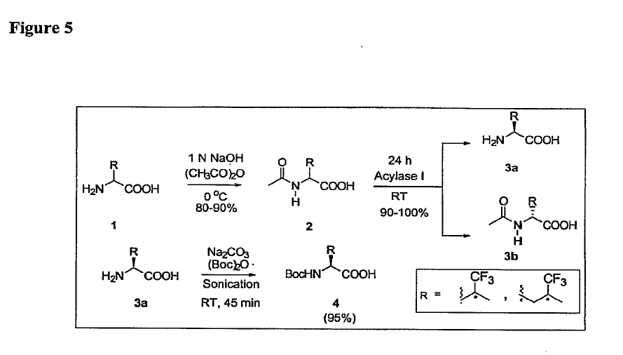

FIG. 6 depicts hemolytic activities of peptides against type B hRBCs relative to melittin. Each data point [M2 (◯), M2F2 (), M2F5 (▪), BII5 (Δ), BII5F2 (▴), BII1 (∇), BII1F2 (▾) and melittin (♦)] is the average of at least two independent experiments with two replicates.

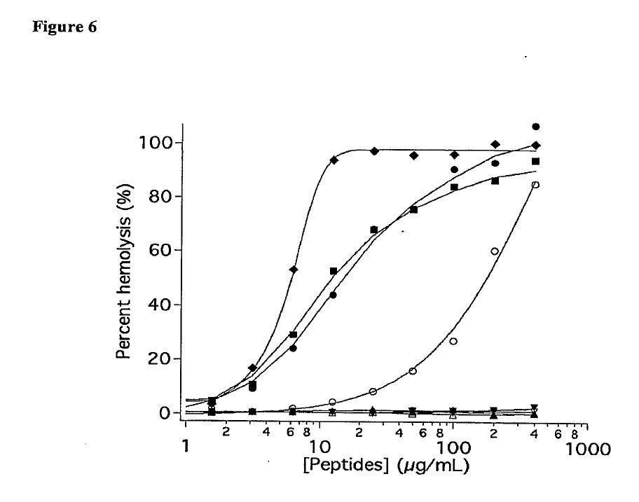

FIG. 7 depicts representative equilibrium analytical ultracentrifugation traces for M2 (A) and M2F5 (B) [25° C., 35 000 rpm at 230 nm]. Fits to a single ideal single species model are shown as a solid line with residuals in the top frame. Conditions: [peptide]=50 μM, 10 mM phosphate, pH 7.40, 137 mM NaCl, 2.7 mM KCl. The observed apparent molecular weights were 2413 (M2, calc. 2478 for monomer) and 12436 (M2F5, calc. 12460 for tetramer). Linear plot of ln(A) vs. r² for M2 (C) indicates a single ideal species while non-random residuals for M2F5 (D) indicate that other aggregation states might be present.

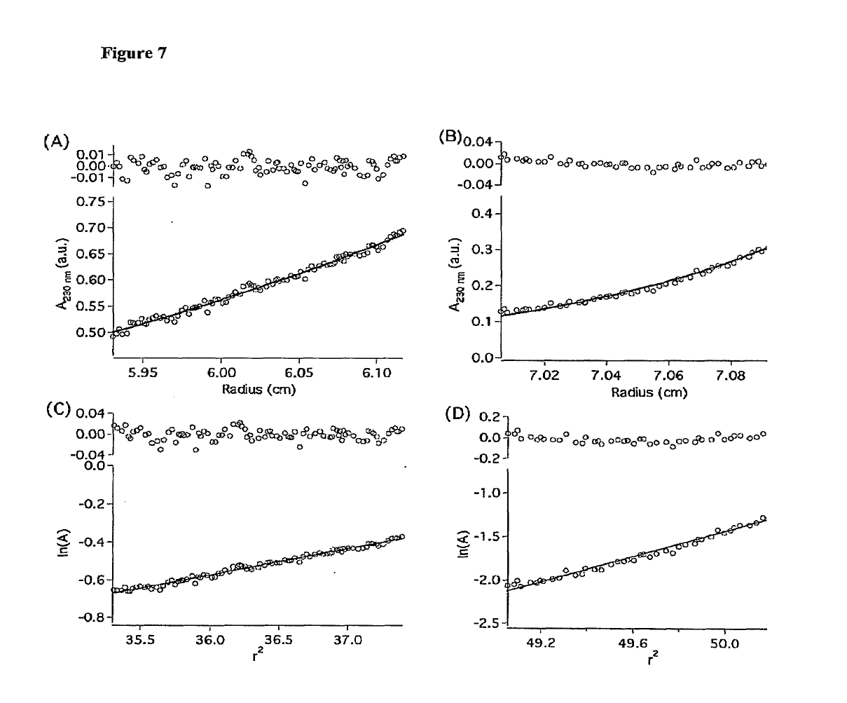

FIG. 8 depicts an HPLC analysis of tryptic mixtures of M2.

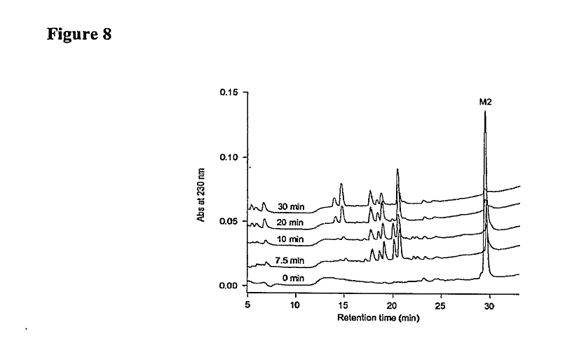

FIG. 9 depicts an HPLC analysis of tryptic mixtures of M2F2.

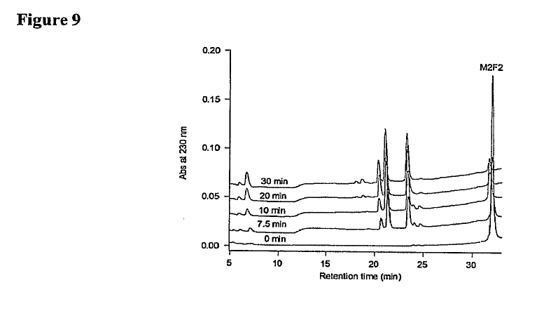

FIG. 10 depicts an HPLC analysis of tryptic mixtures of BII1.

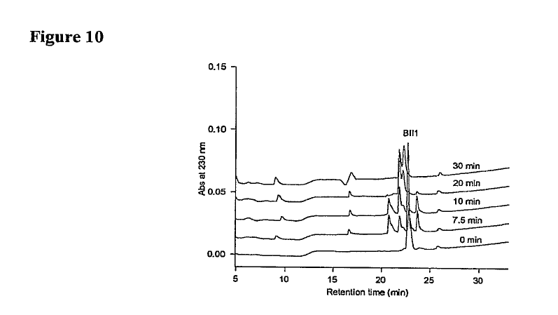

FIG. 11 depicts an HPLC analysis of tryptic mixtures of BII1 F2.

FIG. 12 depicts an HPLC analysis of tryptic mixtures of BII5.

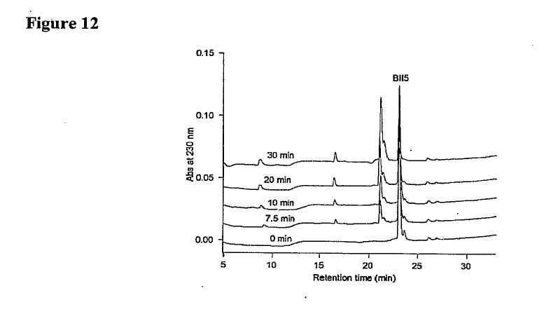

FIG. 13 depicts an HPLC analysis of tryptic mixtures of BII5 F2.

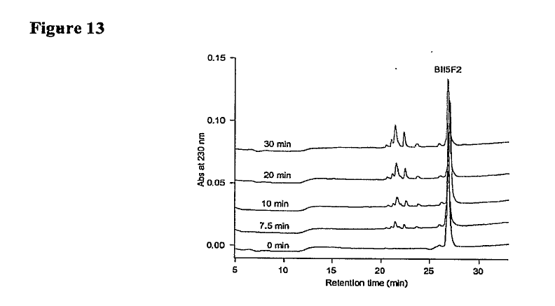

FIG. 14 contains Table 2 which provides the identification of proteolyzed fragments of M2 by ESI-MS.

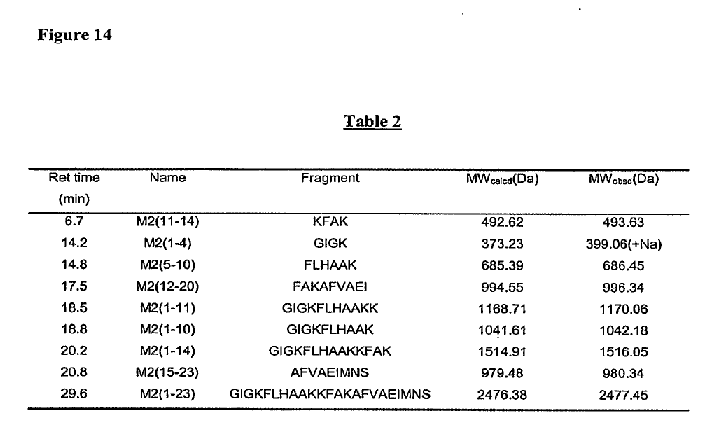

FIG. 15 contains Table 3 which provides the identification of proteolyzed fragments of M2F2 by ESI-MS.

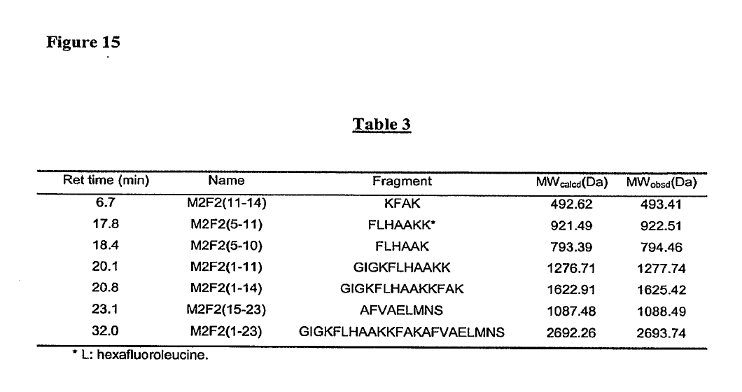

FIG. 16 contains Table 4 which provides the identification of proteolyzed fragments of BII5 by ESI-MS.

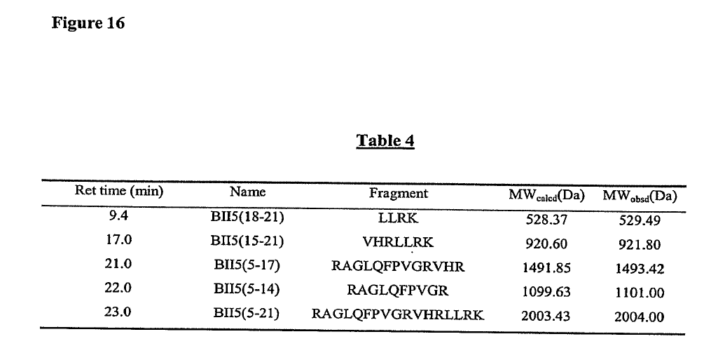

FIG. 17 contains Table 5 which provides the identification of proteolyzed fragments of BII5F2 by ESI-MS.

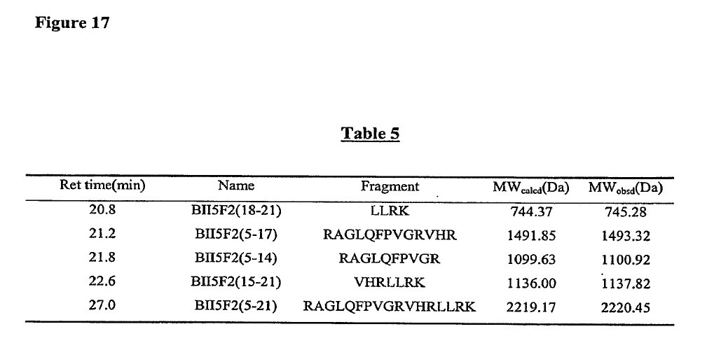

FIG. 18 contains Table 6 which provides the identification of proteolyzed fragments of BII1 by ESI-MS.

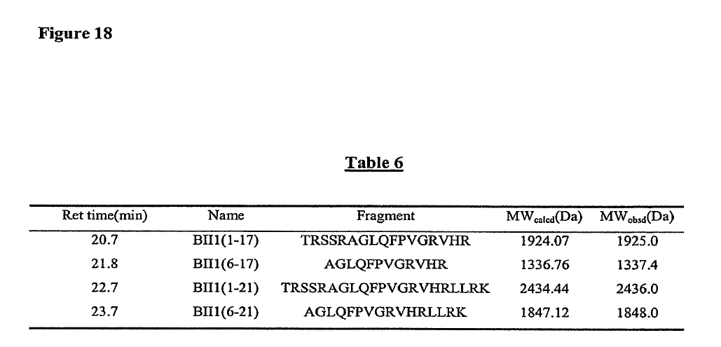

FIG. 19 contains Table 7 which provides the identification of proteolyzed fragments of BII1 F2 by ESI-MS.

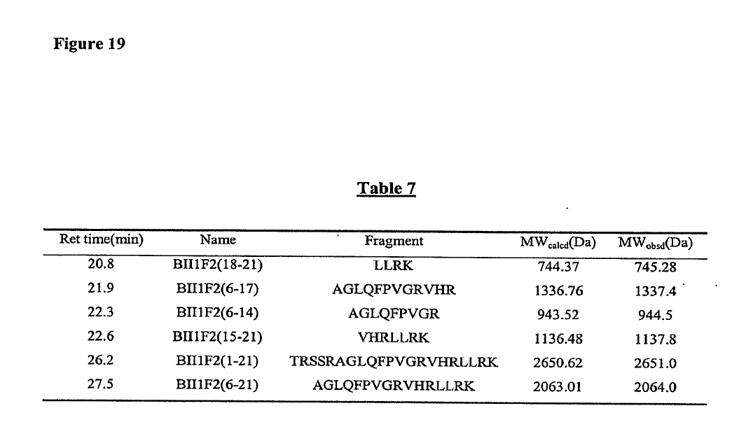

FIG. 20 contains Table 8 which provides initial pseudo-first order rate constants from protease cleavage.

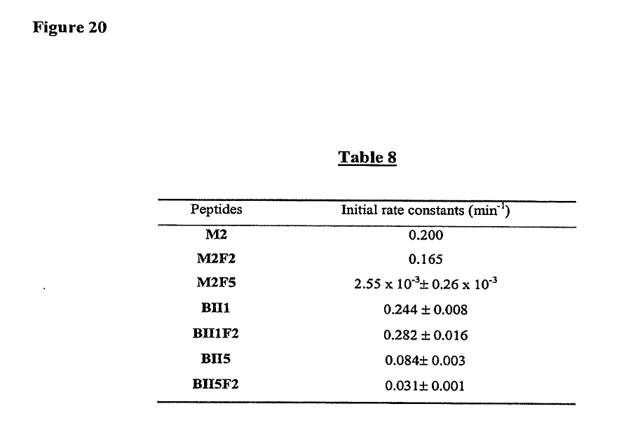

FIG. 21 depicts the kinetics of protease action (trypsin) as probed using analytical RP-HPLC. Degradation of full-length peptides in M2 series (A) and BII series (B). The data represent the average of two independent experiments and are shown with standard deviations. The data were fit using an exponential decay function using Igor Pro v 5.03.

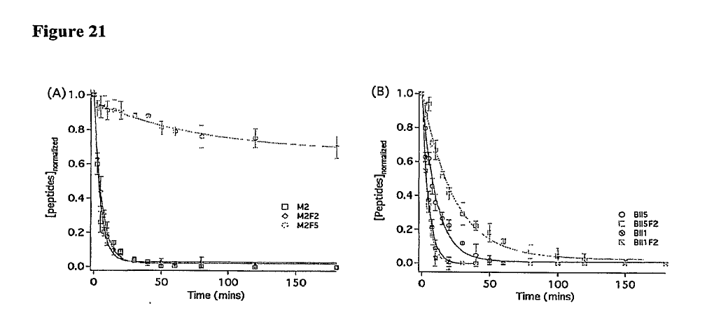

FIG. 22 depicts an HPLC trace of reaction mixture after incubation for 24 h of M2F5 with trypsin at 37° C.

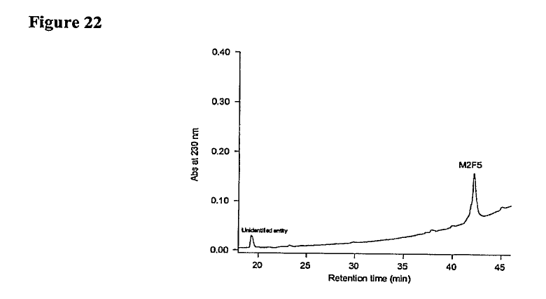

FIG. 23 depicts the concentration of digested fragments from BII5 and BII5F2 released as a function of time. The y-axis is integration area at 230 nm under the peak.

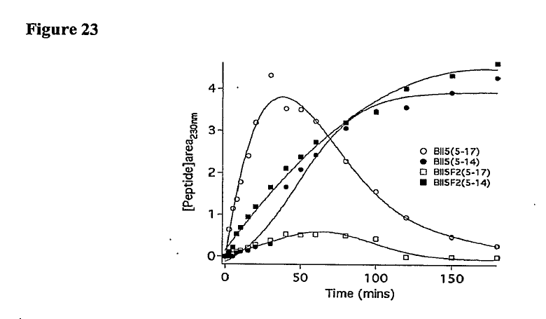

FIG. 24 depicts circular dichroism (CD) data at a number of concentrations of TFE (M2).

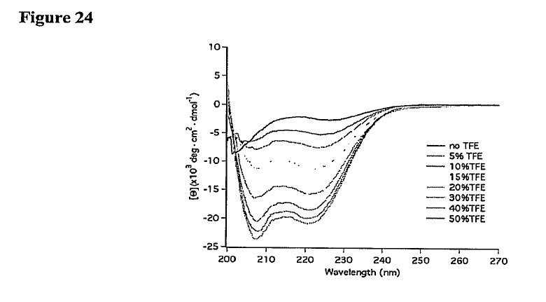

FIG. 25 depicts CD data at a number of concentrations of TFE (M2F2).

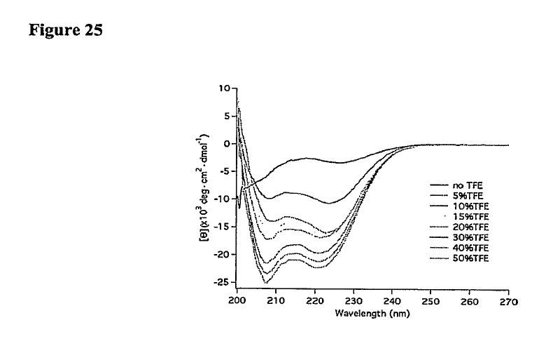

FIG. 26 depicts CD data at a number of concentrations of TFE (M2F5).

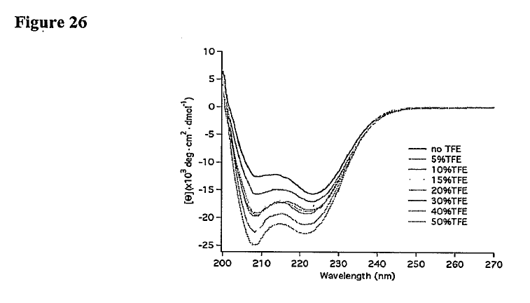

FIG. 27 depicts effect of TFE on helical content of M2, M2F2 and M2F5.

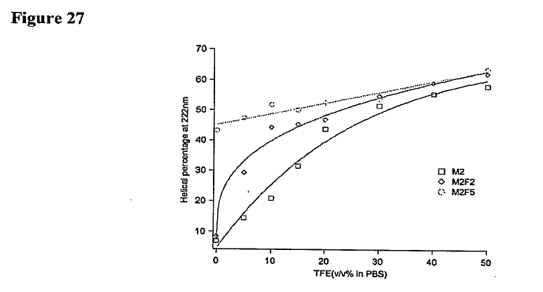

FIG. 28 depicts CD data at a number of concentrations of TFE (BII1).

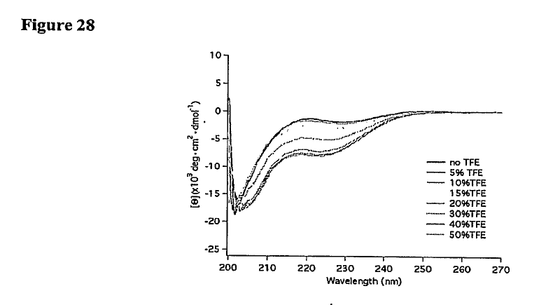

FIG. 29 depicts CD data at a number of concentrations of TFE (BII1F2).

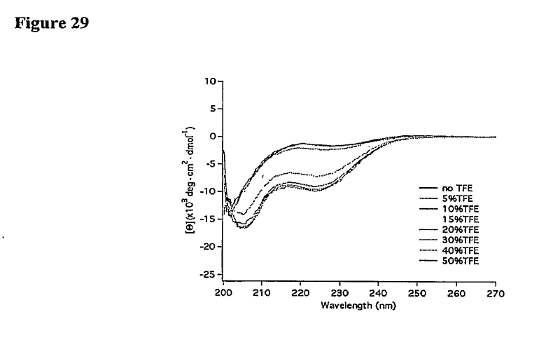

FIG. 30 depicts CD data at a number of concentrations of TFE (BII5).

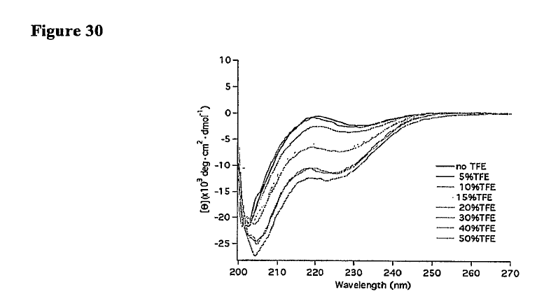

FIG. 31 depicts CD data at a number of concentrations of TFE (BII5F2).

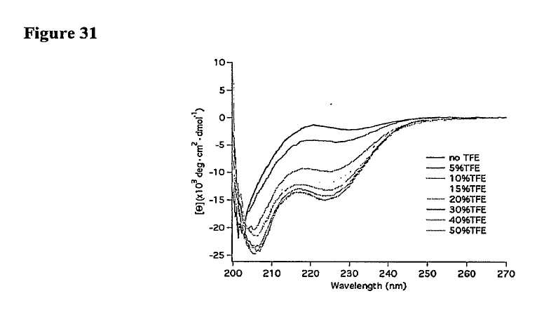

FIG. 32 contains Table 9 which provides apparent molecular weights determined by equilibrium sedimentation. All samples are in 10 mM phosphate pH 7.4, 137 mM NaCl, 2.7 mM KCl.

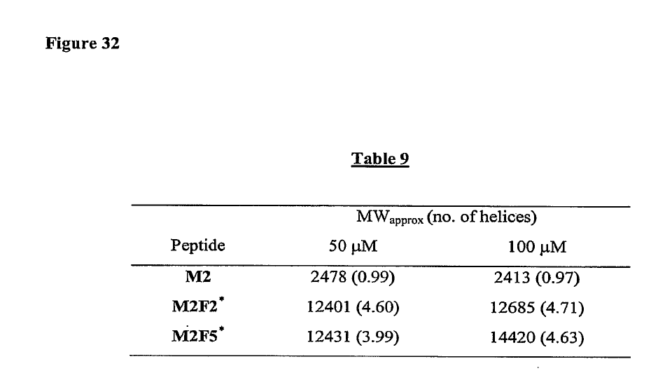

FIG. 33 depicts the hemolytic activity of all antimicrobial peptides was measured against fresh human red blood cells (type B) in two independent experiments (except for M2F5) in duplicate. The melittin and PBS buffer serve as positive and negative control, respectively. The data represent mean±s.d.

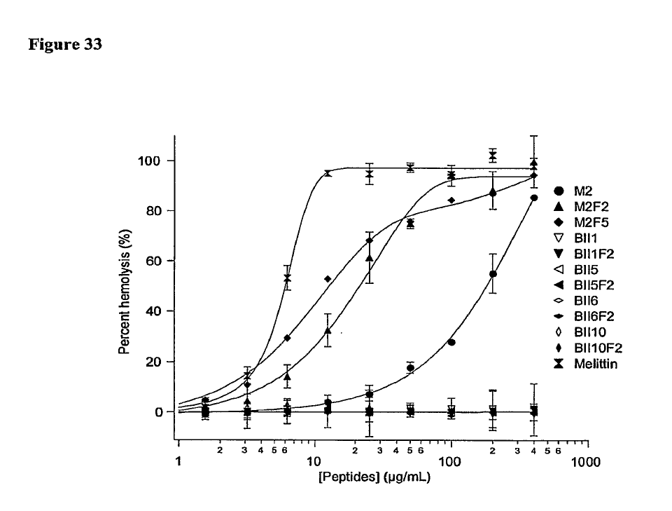

FIG. 34 contains Table 10 which provides minimal inhibitory concentrations (MIC) against E. coli and B. subtilis and percentage hemolysis values for all peptides (^{a} Values are the median of at least two independent experiments done in duplicate;^{b} Percentage hemolysis relative to melittin (100-400 μg/mL)). MIC values have an error factor of 2.

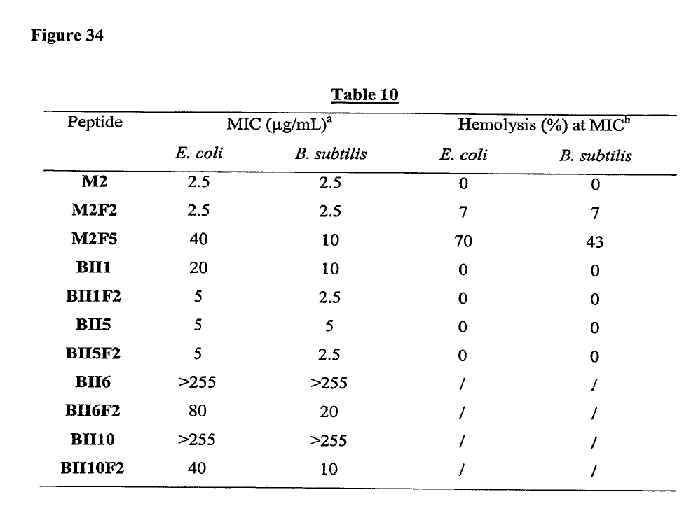

FIG. 35 depicts the sequences of wild type GLP-1 (7-36) amide, fluorinated analogs, exendin (9-39), and [¹²⁵I]-exendin (9-39). All peptides were C-terminally amidated and the residues replaced were underlined. Red arrow indicates the scissile bond subjective to DPP IV. [¹²⁵I]-exendin (9-39) amide was employed as radioligand for the competition binding assay and the conserved residues relative to wild type GLP-1 were colored blue. L represents 5,5,5,5′,5′,5′-2S-hexafluoroleucine and the crystal structure of hexafluoroleucine methyl ester is shown at bottom right.

FIG. 36 depicts binding of peptides to the human GLP-1R expressed on COS-7 cells examined by competitive binding assay using [¹²⁵I]-Ex (9-39) as radioligand. Data represent five independent experiments in duplicate (mean±s.e.m).

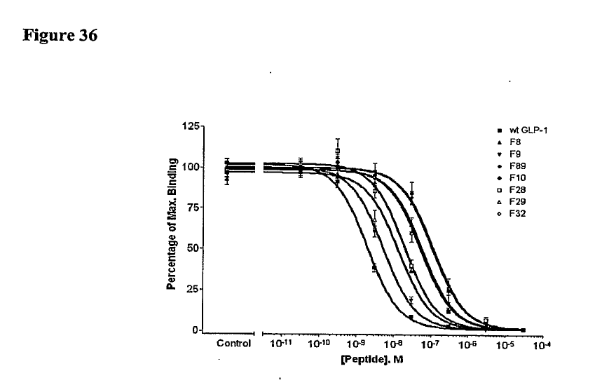

FIG. 37 depicts cAMP production stimulate by wt GLP-1 and fluorinated analogs. Data represent at least three to five independent experiments in duplicate as mean±s.e.m.

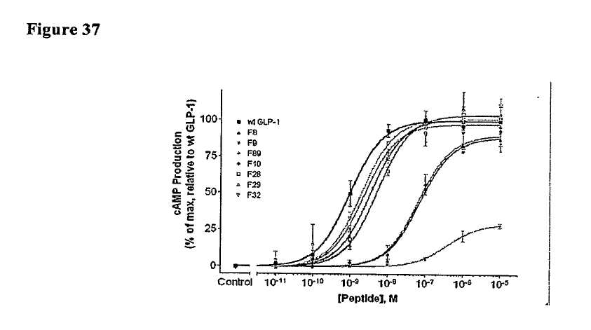

FIG. 38 depicts A) Rate constants of peptide degradation by DPP IV in 50 mM Tris HCl, 1 mM EDTA, pH 7.6, error bars represent standard deviations. [Peptide]=10 μM. [DPP IV] 20 U/L; B) RP-HPLC traces of F8. P1, P2, and P3 denote the F8 at 0, 48 h at [DPPIV]=20 U/L, and 1 h at [DPPIV]=200 U/L; and C) RP-HPLC traces of F89. P1′, P2′, and P3′ denote that F89 at 0, 20, and 60 mins. No detectable hydrolysis products for both F8 and F89 degradation using DPP IV. The traces were offset at x-axis for clearance.

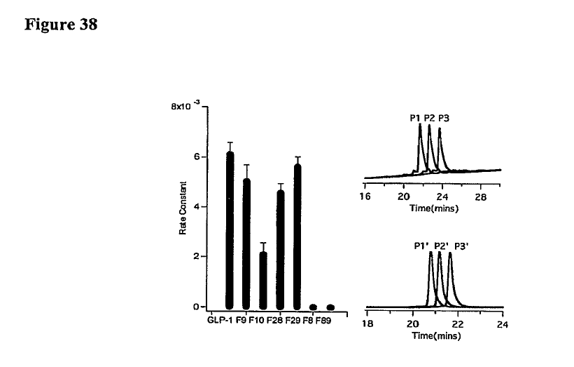

FIG. 39 contains Table 11 which provides a summary of the receptor binding, cAMP production and enzymatic stability of wild type GLP-1 and fluorinated analogs.

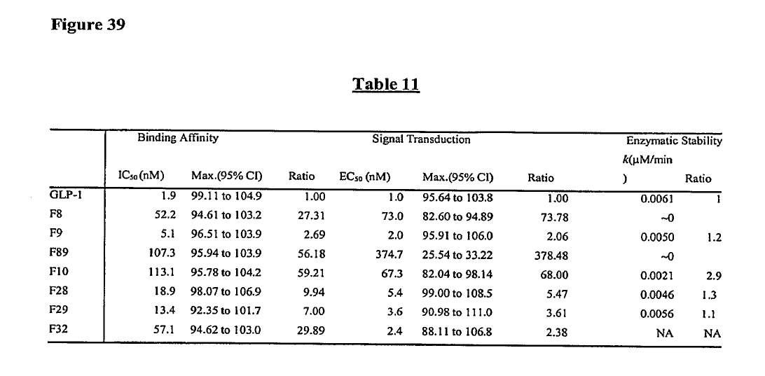

FIG. 40 depicts an OGTT experiment carried out according to protocols and guidelines established by the Tufts IACUC. Normal male mice (C57BL/6), 7-8 weeks of age, were purchased from Charles River Labs, housed in groups of five, with a 12 h light: 12 h darkness cycle. Food was withdrawn for a 20 h period prior to i.p. injection (time −30 min) of PBS as negative control, GLP-1, and fluorinated peptides (30 mmol/kg) in PBS, pH 7.4. All injections were performed at a final volume of 10 ml/kg body weight. At time 0 min, the mice received sterile glucose solution (50% w/v) through oral gavage at a dose of 5 g/kg body weight. Subsequent blood glucose concentration was measured through the tail vein using a OneTouch glucose meter in duplicate at 15, 30, 60, and 120 min. The data were expressed as mean±s.e.

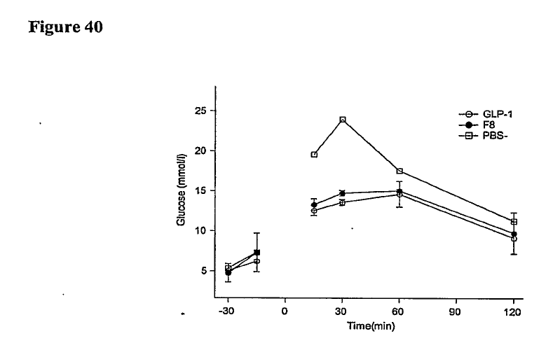

FIG. 41 depicts a comparison of the weights of treated mice. All mice (6) were alive five days post-treatment (Dec. 19, 2006); their weights are compared with those on the treatment day (Dec. 14, 2006). The weight error is approximately ±0.1 g.

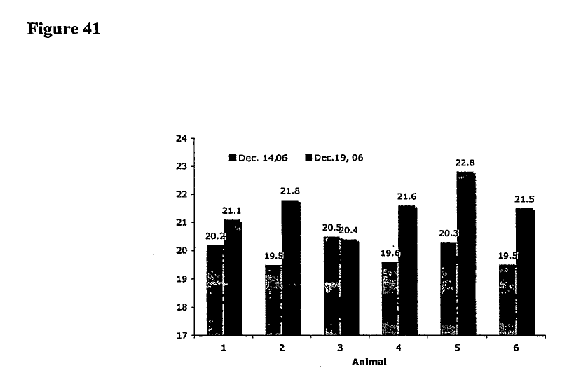

FIG. 42 depicts the set of experiments performed with a final dose of peptides at 3 mmol/kg. Other conditions were the same as that described for FIG. 40. The D-glucose solution was freshly prepared and filtrated with a 0.2 μM filter.

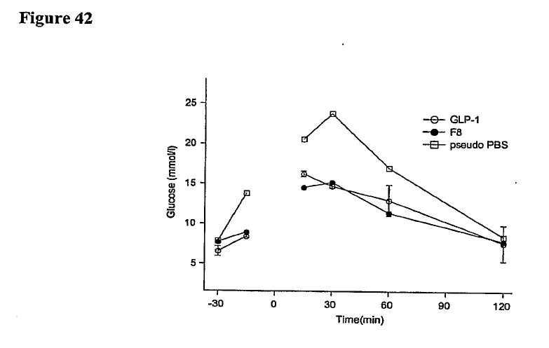

### DETAILED DESCRIPTION OF THE INVENTION

### Antimicrobial Activity and Protease Stability of Proteins Comprising Fluorinated Amino Acids

Peptides were synthesized manually using the in-situ neutralization protocol for t-Boc chemistry on a 0.075 mmol scale with MBHA and Boc-lys (2-Cl-Z)-Merrifield resins. The dinitrophenyl protecting group on histidine was removed using a 20-fold molar excess of thiophenol. Peptides were cleaved from the resin by treatment with HF/anisole (90:10) at 0° C. for 2 h and then precipitated with cold Et₂O. Crude peptides were purified by RP-HPLC [Vydac C₁₈, 10 μM, 10 mm×250 mm]. The purities of peptides were more than 95% as judged by analytical RP-HPLC [Vydac C₁₈, 5 μM, 4 mm×250 mm]. The molar masses of peptides were determined MALDI-TOF MS. Peptide concentrations were determined by quantitative amino acid analysis.

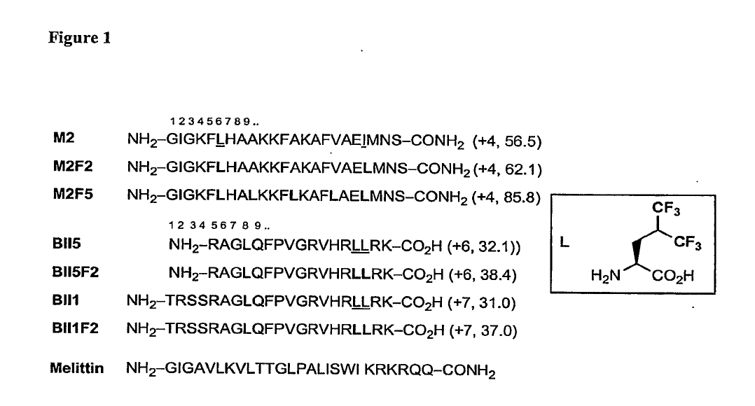

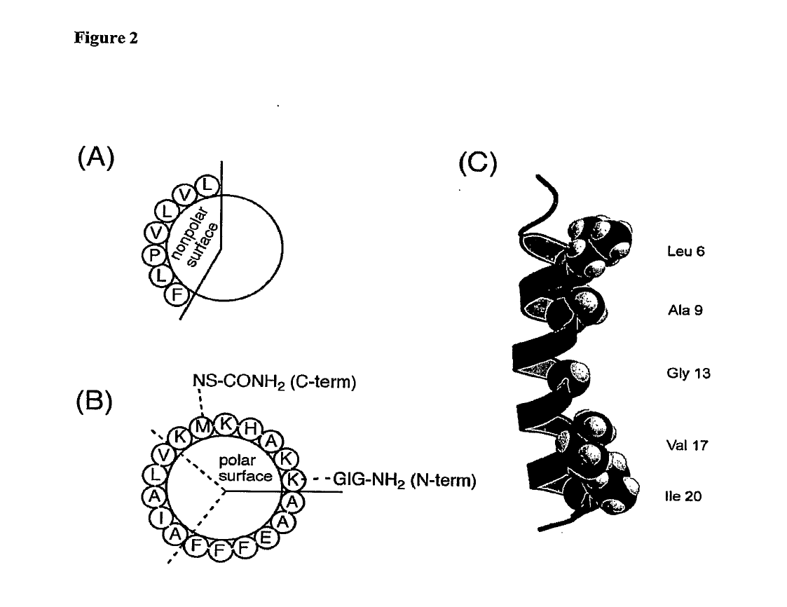

M2 (SEQ ID NO 1) and buforin II[1-21] (BII1) (SEQ ID NO 2), two of the most potent antimicrobial peptides known, were chosen as templates for fluorination. While both peptides are capable of exerting their bactericidal activity at low micromolar concentrations, their modes of action are quite distinct. Although both are initially drawn to negatively charged bacterial membranes by electrostatic interactions, M2 causes cell lysis by forming torodial pores in lipid bilayers, while BII1 penetrates into the cell and kills bacteria by binding intracellular DNA and RNA. Both pore formation and translocation of BII1 into cells seem to be controlled by hydrophobic interactions. We envisaged that incorporation of the super-hydrophobic hexafluoroleucine at selected positions would simultaneously increase membrane affinity and provide greater protease stability. A third template, BII5 (SEQ ID NO 3) employed in our study was an N-terminal truncated buforin II(5-21) that has higher antimicrobial activity compared to Bill. The sequences of peptides and the fluorinated analogues are shown in FIG. 1. Since these peptides adopt amphipathic helical conformations, sites of fluorination were selected on the nonpolar face of helices with the help of helical wheel diagrams (FIG. 2).

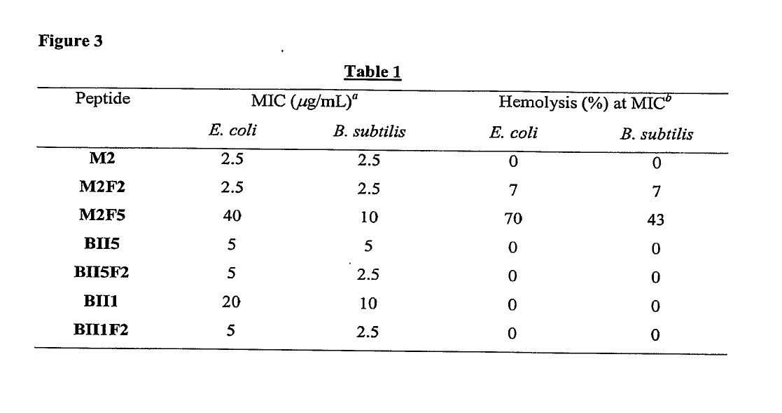

The antimicrobial activity was assessed as a minimal inhibitory concentration (MIC) using turbidity assays against both Gram-positive (B. subtilis) and Gram-negative (E. coli) bacteria (FIG. 3). All fluorinated peptides have comparable or more potent antimicrobial activities relative to the parent peptides with the exception of M2F5. M2F2 exhibited similar MIC values as M2 and M2F5 is 4- and 16-fold less active against B. subtilis and E. coli respectively. On the other hand, the buforin analogues are at least as potent (BII1F2) or 4-fold more potent (BII5F2) than the respective controls. These data clearly demonstrate that the antimicrobial activity is either retained or enhanced upon fluorination.

The selectivity with which the peptides are able to lyse bacterial cells compared to mammalian cells was interrogated by a hemolysis assay against human red blood cells (hRBC). The two buforin analogues had hemolytic activity essentially the same as that of the control peptides suggesting that passage across the membrane was not compromised by fluorination (FIG. 3, Table 1). M2F2 was slightly more hemolytic than M2, whereas M2F5 was significantly more hemolytic than the parent peptide. It has been demonstrated previously that increased hydrophobicity correlates with hemolytic activity. Our results are consistent with this trend. These data point to a maximum hydrophobicity of the parent peptide (>75% Solvent B required for elution in RP-HPLC under the conditions specified in FIG. 1) beyond which fluorination may not result in retention of selectivity for bactericidal activity over mammalian cell permeabilization.

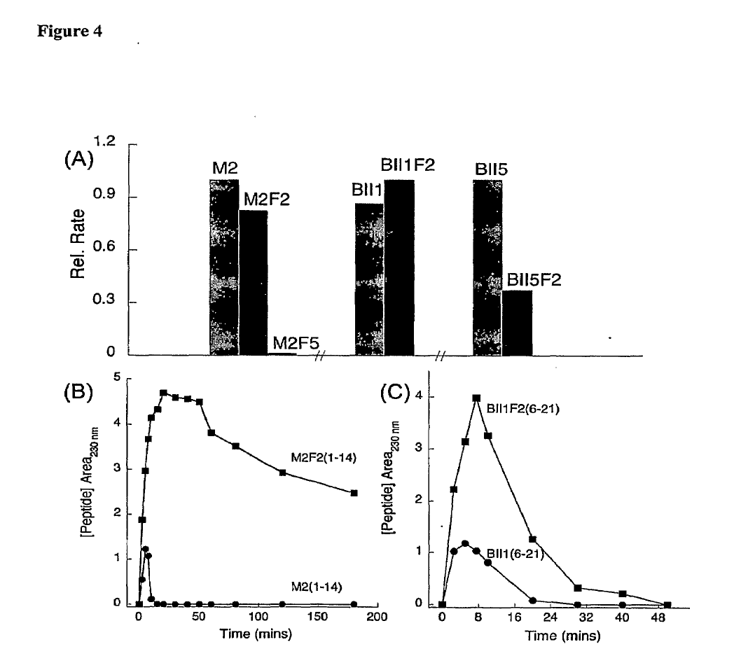

The cationic peptides used in this study were tested for cleavage by trypsin, which catalyzes hydrolysis of C-terminal amide bonds of lysine and arginine. All fluorinated peptides were similar or more stable to proteases (FIG. 4). The buforin II analogue BII5F2 was ˜3 fold more resistant to hydrolysis, while BII1F2 was similar to BII1. Furthermore, the initial P1 site of cleavage was different in BII1F2 (R14) than BII1 (R17). In addition, the initial cleavage fragment BII1F2 (6-21) accumulated and persisted much longer than BII1 (6-2 1). In both cases, the presence of hexafluoroleucine at the P1′ and P2′ sites seems to confer protection to the R17 cleavage site. A similar trend was observed for the magainin analogues. M2F2 was more stable to proteolysis by a factor ˜1.2 relative to M2, whereas M2F5 was fiercely resistant to degradation, with >78% of the peptide remaining in solution after 3 h. In contrast, M2 is completely hydrolyzed in 33 mins. The initial fragment resulting from cleavage, M2F2 (1-14) accumulated in higher amounts than M2 (1-14) and only underwent minimal proteolytic degradation over 3 h.

The presence of a single hexafluoroleucine residue (P2′ site) at position 6 in M2F2 (1-14) confers a dramatic advantage in protecting the K4 amide bond. Unlike fluoromethylketone or β-fluoro α-keto ester and acid terminated peptides, the fluorine substitution in this instance is not proximal to the hydrolysis site. While an electronic perturbation may still be operational, it is more likely that the protease protection is a result of steric occlusion of the peptide from the active site or because of increased conformational stability of folded entities that deny protease access to the labile amide.

Circular dichroism (CD) spectroscopy was used to probe secondary structure. All peptides with the exception of M2F5 were random coil in aqueous solutions. However, with increasing amounts of trifluoroethanol (TFE), the peptides adopted an α-helical structure. At 50% TFE, both M2 and M2F2 were ˜60% helical. In contrast, M2F5 was helical to the same extent in buffered aqueous solutions with no TFE. Furthermore, M2 was monomeric as judged by analytical ultracentrifugation while both M2F2 and M2F5 had a tendency to populate multiple oligomeric states. Indeed, M2F5 appears to form helical bundles providing an explanation for both decreased antimicrobial activity and greatly enhanced protease stability.

### Influence of Selective Fluorination of GLP-1 on Proteolytic Stability and Biological Activity

Peptide Design. GLP-1 binds to the GLP-1R on the pancreatic β cells and the hydrophobic interactions are likely the major driving force responsible for the association of this amphiphilic α-helical peptide to its receptor. Structural studies on GLP-1 both in a dodecylphosphate choline micelle and in 35% TFE by 2D NMR showed that GLP-1 consists of a N-terminal random coil segment (7-13), two helical segments (13-20 and 24-37), and a linker region (21-23). The C-terminal helix is more stable than the N-terminal helix determined by amide proton exchange experiments and was an essential contributor of binding to GLP-1R. Replacements of Phe²⁸ and Ile²⁹ to alanine led to the dramatic lose of the binding affinity to GLP-1R. These two residues along with Trp³¹, Leu³², Gly³⁵ are conserved between GLP-1 and exendin 4, a synthetic GLP-1R agonist with high affinity and are located on the C-terminal hydrophobic surface. In an attempt to improve the binding affinity of GLP-1 to GLP-1R, Phe²⁸, Ile²⁹ and Leu³² were selectively substituted by hexafluoroleucine under the consideration that increased hydrophobicity of hexafluoroleucine would possibly lead to an enhanced binding affinity. The Trp³¹ was kept unchanged not only because this chromophore will be used for determining the peptide concentration but also it has a large side chain volume. The Gly³⁵ was also remained since the flexibility it provided has been proposed essential for the receptor binding.

To render the resistance towards DPP IV, the primary enzyme for the rapid deactivation of GLP-1, the N-terminal residues (P1, P1′ and/or P2′ positions) were substituted by hexafluoroleucine, namely, Ala⁸, Glu⁹, Gly¹⁰ and both Ala⁸ and Glu⁹ to generate four fluorinated analogs. The His⁷ was kept unchanged since its particularly crucial role for sending signal to the receptor.

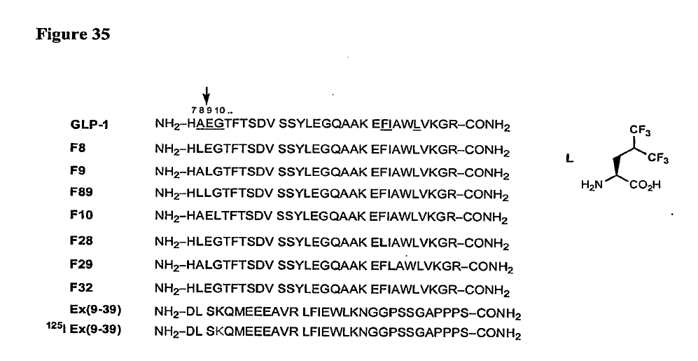

In short, the N-terminal replacements were aimed to enhance enzymatic stability and the C-terminal substitutions were intended to test fluorination effect on binding affinity to receptor. The total seven-fluorinated analogs, the wild type GLP-1, and [¹²⁵I]-exendin (9-39) amide are listed in FIG. 35.

Binding Assay. The binding affinity of fluorinated analogs was measured by a competition-binding assay using [¹²⁵I]-exendin (9-39) amide as a radioligand. This Bolton-Hunter labeled peptide was assumed to have a similar affinity to hGLP-1R as exendin (9-39) amide since the modification at Lys¹² side-chain does not damage the receptor binding. The homologous antagonist competitive binding experiments showed that the binding of exendin (9-39) amide has a dissociation constant of 2.9 nM (three independent experiments in triplicate), comparable to previous reported data. All 7 fluorinated GLP-1 analogs bound to the hGLP-1R expressed on COS-7 cells, which lack of endogenous GLP-1R. F9 had a 2.7-fold decreased binding affinity compared to wt GLP-1 (IC₅₀ 5.1 nM vs 1.9 nM, FIG. 1 and Table 1), while F29 and F28 displayed 7-fold and 9.9-fold decreased affinity. F8, F89, F10, and F32 lost the binding affinity by 27˜60 fold. The carboxylate of Glu⁹ has been proved important for the receptor binding as substitution by Lys⁹ resulted in a dramatic lose in terms of binding affinity. Its substitution by Ala⁹ led to relatively poor receptor binding (30˜100-fold higher IC₅₀), while substitution by Asp⁹ did not exhibit remarkable changes in receptor binding (about same IC₅₀). These facts, together with the similar binding affinity showed by F9, Glu⁹ was replaced by hexafluoroleudcine, led to a plausible explanation that the “polar hydrophobicity” of hexafluoroleucine is probably responsible for the no apparent lose of binding affinity or the bulky hydrophobic side chains at this position are well tolerated. These data here indicate that fluorination led to a slightly to moderate decrease of binding affinity to GLP-1R. The N-terminal modifications, except for F9, resulted in pronounced decrease of binding affinity, while the C-terminal modifications were well tolerated.

Formation of cAMP. To examine whether the fluorinated analogs remain to be functional as full agonists, partial agonists or antagonists, COS-7 cells with hGPL-1R were stimulated by peptides and the production of cAMP were measured by a radioimmunoassay. All fluorinated peptides remain as full agonists except for F89 and subsequent the dose-response was measured for all peptides (FIG. 2). F9, F32, F29, and F28 had a 2.1, 2.4, 3.6, and 5.4-fold decreased potency while remaining the important efficacy as wt GLP-1 (FIG. 2 And Table 1). F8 and F10 showed moderate 68 and 73.8-fold lower potency with slightly decreased the efficacy, which were not statistically significant byp-test. Unexpected, F89 turned out to be a partial agonist and had a dramatic decrease of potency, 378-fold lower than wt GLP1, while conserving the similar binding ability to receptor as F10 in the range of tested concentrations. Since the histidine residue of N-terminal random coil is responsible for initiating the signal to the receptor, the change of the secondary structure at this portion may have apparent influence on the stimulation of cAMP production. Or, the side chains of hexafluoroleucine disturb the receptor conformational change. Overall, analogs with a lower receptor affinity were, by and large, exhibited a higher EC₅₀ value with respect to activation of adenylyl cyclase.

Proteolytic Stability. Wt GLP-1 is rapidly inactivated by ubiquitous enzyme DPP IV, setting the obstacle up for native GLP-1 as a therapeutic agent (in human t_{1/2}≈1˜3 mins). DPP IV has a relative specific requirement for substrate residues at P2, P1, P1′ and P2′ positions regarding the scissile Ala-Glu amide bond. Especially, at P1 position, Pro and Ala are highly favored. In contrast, other amino acids and derivatives at this 8 position enhanced the peptide stability, as the reported case Gly⁸, Aib⁸, Ser⁸, Thr⁸, Leu⁸. From our previous studies, incorporation of hexafluoroleucine close to the scissile bond is able to modulate the resistance of peptides towards hydrolytic protease. Under the selected experimental conditions, as expected, replacement by hexafluoroleucine at 8, 9, 10 positions endowed DPP IV resistance to different extent. F8 and F89 showed dramatic resistance as no fragments were detected after 24 h incubation. To further examine the stability, FS was incubated with DPP IV at a 10-fold higher concentration, no fragments were detected after 1 h. F9 and F10 exhibited ˜1.2-fold and 2.9-fold resistance by comparing the initial first-order rate constants (FIG. 3), and HPLC analysis showed the formation of only one other major peak, which was identified by ESI-MS as corresponding peptide fragment GLP-1 (9-36) amide. The kinetic data reported here for the fluorinated GLP-1 analogs could plausiblely correlate to the prolonged metabolic stability in vivo, which has been established by Deacon et. al. In their study, daily administration of Val⁸-GLP-1 resulted in the increased insulin level and reduced plasma glucose more than wt GLP-1. Taken together, F8, F9, F10, and F29 showed promising potential as candidates for further animal glucose tolerance study.

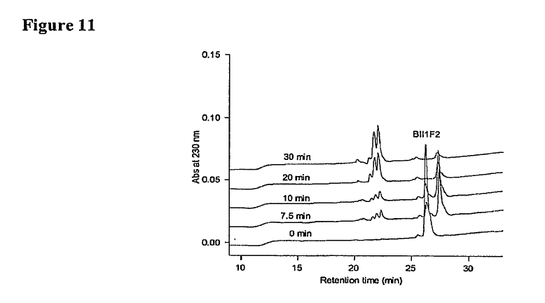

As seen in FIG. 11, both enzymatic kinetic studies on GLP-1 analogs with mutation at position 8 and the X-ray crystal structural investigation of human DPP IV with a decapeptide substrate or an inhibitor show that the enzyme demands an amino acid with a small side to chain at 8 position to fit in the binding pocket. While the hexafluoroleucine (bearing a large side chain functionality) was incorporated at N-terminal modifications, the resistance against DPP IV was observed. The result here is in good agreement with previous kinetic and structural studies. The F9 and F10 containing hexafluoroleucine at P1′ and P2′ positions also displayed moderate enhanced resistance to DPP IV. In contrast to other methodologies employed for prolonging the half-life time of therapeutic peptides/proteins, such as pegylation, glycosylation, and conjugation to serum protein albumin, incorporation of fluorinated amino acid clearly proves their potential usages especially when small peptides are the targets to be modified as these non-natural amino acids can be rapidly incorporated by solid phase peptide synthesis. The changes of potency of fluorinated analogs could be due to slightly structural variations at the N-terminal random region. The C-terminal modifications were motivated to enhance binding affinity to the receptor, which were not achieved; rather, slightly decreased binding affinity was observed. These results may not be surprising since the elegant interactions between GLP-1 and its receptor have evolved by nature over million years so that minor structural change of ligand will possibly lead to the decreased affinity of the ligand. However, this lock-and-key type interaction could be strengthened by design if detailed structural information of ligand and receptor is available, or by a large library screening.

Thus alternations in the N-terminus of GLP-1 with hexafluoroleucine confer DPP IV resistance while retaining the biological activity in terms of in vitro efficacy, suggesting that using fluorinated amino acids is a promising methodology to make bioactive peptides more metabolically stable with a retain and only slightly decreased biological activity (FIG. 11).

### Definitions

For convenience, certain terms employed in the specification, examples, and appended claims are collected here.

The term “heteroatom” as used herein means an atom of any element other than carbon or hydrogen. Preferred heteroatoms are boron, nitrogen, oxygen, phosphorus, sulfur and selenium.

As used herein, the definition of each expression, e.g., amino acid, m, n, etc., when it occurs more than once in any structure, is intended to be independent of its definition elsewhere in the same structure.

The phrase “protecting group” as used herein means temporary substituents which protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed (Greene, T. W.; Wuts, P. G. M. Protective Groups in Organic Synthesis, 2^{nd} ed.; Wiley: New York, 1991).

As used herein, “natural” or “wild type” refers to a protein or a polypeptide, which is found in nature, and “artificial” refers to a protein or a polypeptide that comprises non-natural sequences and/or amino acids. The term “amino acid” is used herein in its broadest sense, and includes naturally occurring amino acids as well as non-naturally occurring amino acids, including amino acid analogs and derivatives. The latter includes molecules containing an amino acid moiety. One skilled in the art will recognize, in view of this broad definition, that reference herein to an amino acid includes, for example, naturally occurring proteogenic L-amino acids; D-amino acids; chemically modified amino acids such as amino acid analogs and derivatives; naturally occurring non-proteogenic amino acids, and chemically synthesized compounds having properties known in the art to be characteristic of amino acids.

As used herein, the term “non-natural amino acid” refers to an amino acid that is different from the twenty naturally occurring amino acids (alanine, arginine, glycine, asparagine, aspartic acid, cysteine, glutamine, glutamic acid, serine, threonine, histidine, lysine, methionine, proline, valine, isoleucine, leucine, tyrosine, tryptophan, phenylalanine) in its side chain functionality.

The term “hydrophobic” when used in reference to amino acids refers to those amino acids which have nonpolar side chains. Hydrophobic amino acids include valine, leucine, isoleucine, cysteine methionine, phenylalanine, tyrosine and tryptophan.

As used herein, the term “fluorinated amino acid” refers to an amino acid that differs from the naturally occurring amino acid via incorporation of fluorine in place of one or more hydrogens in its side chain functionality. Exemplary fluorinated amino acids may include trifluoroleucine, 4,4,4-trifluorovaline, 5,5,5-trifluoroleucine, trifluorovaline, hexafluorovaline, trifluoroisoleucine, trifluoronorvaline, hexafluoroleucine, 5,5,5,5′,5′,5′-hexafluoroleucine, trifluoromethionine, trifluoromethylmethionine and fluorophenylalanine.

The term “polypeptide” when used herein refers to two or more amino acids that are linked by peptide bond(s), regardless of length, functionality, environment, or associated molecule(s). Typically, the polypeptide is at least four amino acid residues in length and can range up to a full-length protein. As used herein, “polypeptide,” “peptide,” and “protein” are used interchangeably.

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

For purposes of this invention, the chemical elements are identified in accordance with the Periodic Table of the Elements, CAS version, Handbook of Chemistry and Physics, 67th Ed., 1986-87, inside cover.

When used herein, the term “biologically active” refers to an ability to exhibit a biological function.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

The term “treating” refers to: (i) preventing a disease, disorder or condition from occurring in an animal which may be predisposed to the disease, disorder and/or condition but has not yet been diagnosed as having it; (ii) inhibiting the disease, disorder or condition, i.e., arresting its development; and (iii) relieving the disease, disorder or condition, i.e., causing regression of the disease, disorder and/or condition.

### METHODS OF THE INVENTION

In certain embodiments, the invention relates to a method for preparing a modified peptide, comprising
(a) identifying a natural or non-natural peptide; and
(b) synthesizing a modified peptide based on the sequence of said natural or non-natural peptide;
wherein at least one amino acid of the natural or non-natural peptide is replaced by at least one fluorinated amino acid in said modified polypeptide; and said modified polypeptide has increased stability relative to said natural or non-natural peptide.

In certain embodiments, the invention relates to the aforementioned method, wherein said increased stability is chemical, thermal, or proteolytic.

In certain embodiments, the invention relates to the aforementioned method, wherein said increased stability is chemical.

In certain embodiments, the invention relates to the aforementioned method, wherein said increased stability is chemical, and said stability is increased by less than or equal to about 15 kcal/mol when measured as ΔΔG°_{unfolding}.

In certain embodiments, the invention relates to the aforementioned method, wherein said increased stability is chemical, and the increase is greater than about 0.1 kcal/mol and less than or equal to about 15 kcal/mol when measured as ΔΔG°_{unfolding}.

In certain embodiments, the invention relates to the aforementioned method, wherein said increased stability is chemical, and the increase is greater than about 0.5 kcal/mol and less than or equal to about 15 kcal/mol when measured as ΔΔG°_{unfolding}.

In certain embodiments, the invention relates to the aforementioned method, wherein said increased stability is chemical, and the increase is greater than about 1 kcal/mol and less than or equal to about 15 kcal/mol when measured as ΔΔG°_{unfolding}.

In certain embodiments, the invention relates to the aforementioned method, wherein said increased stability is chemical, and the increase is greater than about 3 kcal/mol and less than or equal to about 15 kcal/mol when measured as ΔΔG°_{unfolding}.

In certain embodiments, the invention relates to the aforementioned method, wherein said increased stability is chemical, and the increase is greater than about 5 kcal/mol and less than or equal to about 15 kcal/mol when measured as ΔΔG°_{unfolding}.

In certain embodiments, the invention relates to the aforementioned method, wherein said increased stability is chemical, and the increase is greater than about 7 kcal/mol and less than or equal to about 15 kcal/mol when measured as ΔΔG°_{unfolding}.

In certain embodiments, the invention relates to the aforementioned method, wherein said increased stability is chemical, and the increase is greater than about 9 kcal/mol and less than or equal to about 15 kcal/mol when measured as ΔΔG°_{unfolding}.

In certain embodiments, the invention relates to the aforementioned method, wherein said increased stability is chemical, and the increase is greater than about 11 kcal/mol and less than or equal to about 15 kcal/mol when measured as ΔΔG°_{unfolding}.

In certain embodiments, the invention relates to the aforementioned method, wherein said increased stability is thermal.

In certain embodiments, the invention relates to the aforementioned method, wherein said increased stability is thermal, and Tₘ is increased by less than or equal to about 50° C.

In certain embodiments, the invention relates to the aforementioned method, wherein said increased stability is thermal, and Tₘ is increased by greater than about 1° C. and less than or equal to about 50° C.

In certain embodiments, the invention relates to the aforementioned method, wherein said increased stability is thermal, and Tₘ is increased by greater than about 5° C. and less than or equal to about 50° C.

In certain embodiments, the invention relates to the aforementioned method, wherein said increased stability is thermal, and Tₘ is increased by greater than about 10° C. and less than or equal to about 50° C.

In certain embodiments, the invention relates to the aforementioned method, wherein said increased stability is thermal, and Tₘ is increased by greater than about 15° C. and less than or equal to about 50° C.

In certain embodiments, the invention relates to the aforementioned method, wherein said increased stability is thermal, and Tₘ is increased by greater than about 20° C. and less than or equal to about 50° C.

In certain embodiments, the invention relates to the aforementioned method, wherein said increased stability is thermal, and Tₘ is increased by greater than about 25° C. and less than or equal to about 50° C.

In certain embodiments, the invention relates to the aforementioned method, wherein said increased stability is thermal, and Tₘ is increased by greater than about 30° C. and less than or equal to about 50° C.

In certain embodiments, the invention relates to the aforementioned method, wherein said increased stability is thermal, and Tₘ is increased by greater than about 35° C. and less than or equal to about 50° C.

In certain embodiments, the invention relates to the aforementioned method, wherein said increased stability is thermal, and Tₘ is increased by greater than about 40° C. and less than or equal to about 50° C.

In certain embodiments, the invention relates to the aforementioned method, wherein said increased stability is thermal, and Tₘ is increased by greater than about 45° C. and less than or equal to about 50° C.

In certain embodiments, the invention relates to the aforementioned method, wherein said increased stability is proteolytic.

In certain embodiments, the invention relates to the aforementioned method, wherein said increased stability is proteolytic, and said stability is increased by less than or equal to a factor of about 10⁹.

In certain embodiments, the invention relates to the aforementioned method, wherein said increased stability is proteolytic, and said stability is increased by greater than a factor of about 1.1 and less than or equal to a factor of about 10⁹.

In certain embodiments, the invention relates to the aforementioned method, wherein said increased stability is proteolytic, and said stability is increased by greater than a factor of about 2 and less than or equal to a factor of about 10⁹.

In certain embodiments, the invention relates to the aforementioned method, wherein said increased stability is proteolytic, and said stability is increased by greater than a factor of about 4 and less than or equal to a factor of about 10⁹.

In certain embodiments, the invention relates to the aforementioned method, wherein said increased stability is proteolytic, and said stability is increased by greater than a factor of about 10 and less than or equal to a factor of about 10⁹.

In certain embodiments, the invention relates to the aforementioned method, wherein said increased stability is proteolytic, and said stability is increased by greater than a factor of about 50 and less than or equal to a factor of about 10⁹.

In certain embodiments, the invention relates to the aforementioned method, wherein said increased stability is proteolytic, and said stability is increased by greater than a factor of about 10² and less than or equal to a factor of about 10⁹.

In certain embodiments, the invention relates to the aforementioned method, wherein said increased stability is proteolytic, and said stability is increased by greater than a factor of about 10³ and less than or equal to a factor of about 10⁹.

In certain embodiments, the invention relates to the aforementioned method, wherein said increased stability is proteolytic, and said stability is increased by greater than a factor of about 10⁴ and less than or equal to a factor of about 10⁹.

In certain embodiments, the invention relates to the aforementioned method, wherein said increased stability is proteolytic, and said stability is increased by greater than a factor of about 10⁵ and less than or equal to a factor of about 10⁹.

In certain embodiments, the invention relates to the aforementioned method, wherein said increased stability is proteolytic, and said stability is increased by greater than a factor of about 10⁶ and less than or equal to a factor of about 10⁹.

In certain embodiments, the invention relates to the aforementioned method, wherein said increased stability is proteolytic, and said stability is increased by greater than a factor of about 10⁷ and less than or equal to a factor of about 10⁹.

In certain embodiments, the invention relates to the aforementioned method, wherein said increased stability is proteolytic, and said stability is increased by greater than a factor of about 10⁸ and less than or equal to a factor of about 10⁹.

In certain embodiments, the invention relates to the aforementioned method, wherein said at least one fluorinated amino acid is selected from the group consisting of trifluoroleucine, 4,4,4-trifluorovaline, 5,5,5-trifluoroleucine, trifluorovaline, hexafluorovaline, trifluoroisoleucine, trifluoronorvaline, hexafluoroleucine, 5,5,5,5′,5′,5′-hexafluoroleucine, trifluoromethionine, trifluoromethylmethionine and fluorophenylalanine.

In certain embodiments, the invention relates to the aforementioned method, wherein said at least one amino acid is leucine; and said at least one fluorinated amino acid is hexafluoroleucine.

In certain embodiments, the invention relates to the aforementioned method, wherein said at least one amino acid is isoleucine; and said at least one fluorinated amino acid is hexafluoroleucine.

In certain embodiments, the invention relates to the aforementioned method, wherein said at least one amino acid is alanine; and said at least one fluorinated amino acid is hexafluoroleucine.

In certain embodiments, the invention relates to the aforementioned method, wherein said at least one amino acid is valine; and said at least one fluorinated amino acid is hexafluoroleucine.

In certain embodiments, the invention relates to the aforementioned method, wherein said at least one amino acid is glycine; and said at least one fluorinated amino acid is hexafluoroleucine.

In certain embodiments, the invention relates to the aforementioned method, wherein said at least one amino acid is glutamic acid; and said at least one fluorinated amino acid is hexafluoroleucine.

In certain embodiments, the invention relates to the aforementioned method, wherein said at least one amino acid is phenylalanine; and said at least one fluorinated amino acid is hexafluoroleucine.

In certain embodiments, the invention relates to the aforementioned method, wherein said at least one amino acid is leucine; and said at least one fluorinated amino acid is trifluoroleucine.

In certain embodiments, the invention relates to the aforementioned method, wherein said at least one amino acid is isoleucine; and said at least one fluorinated amino acid is trifluoroleucine.

In certain embodiments, the invention relates to the aforementioned method, wherein said at least one amino acid is alanine; and said at least one fluorinated amino acid is trifluoroleucine.

In certain embodiments, the invention relates to the aforementioned method, wherein said at least one amino acid is valine; and said at least one fluorinated amino acid is trifluoroleucine.

In certain embodiments, the invention relates to the aforementioned method, wherein said at least one amino acid is glycine; and said at least one fluorinated amino acid is trifluoroleucine.

In certain embodiments, the invention relates to the aforementioned method, wherein said at least one amino acid is glutamic acid; and said at least one fluorinated amino acid is trifluoroleucine.

In certain embodiments, the invention relates to the aforementioned method, wherein said at least one amino acid is phenylalanine; and said at least one fluorinated amino acid is trifluoroleucine.

In certain embodiments, the invention relates to the aforementioned method, wherein said at least one amino acid is leucine; and said at least one fluorinated amino acid is trifluorovaline.

In certain embodiments, the invention relates to the aforementioned method, wherein said at least one amino acid is isoleucine; and said at least one fluorinated amino acid is trifluorovaline.

In certain embodiments, the invention relates to the aforementioned method, wherein said at least one amino acid is alanine; and said at least one fluorinated amino acid is trifluorovaline.

In certain embodiments, the invention relates to the aforementioned method, wherein said at least one amino acid is valine; and said at least one fluorinated amino acid is trifluorovaline.

In certain embodiments, the invention relates to the aforementioned method, wherein said at least one amino acid is glycine; and said at least one fluorinated amino acid is trifluorovaline.

In certain embodiments, the invention relates to the aforementioned method, wherein said at least one amino acid is glutamic acid; and said at least one fluorinated amino acid is trifluorovaline.

In certain embodiments, the invention relates to the aforementioned method, wherein said at least one amino acid is phenylalanine; and said at least one fluorinated amino acid is trifluorovaline.

In certain embodiments, the invention relates to the aforementioned method, wherein said at least one amino acid is glycine; and said at least one fluorinated amino acid is trifluoroisoleucine.

In certain embodiments, the invention relates to the aforementioned method, wherein said at least one amino acid is glutamic acid; and said at least one fluorinated amino acid is trifluoroisoleucine.

In certain embodiments, the invention relates to the aforementioned method, wherein said at least one amino acid is phenylalanine; and said at least one fluorinated amino acid is trifluoroisoleucine.

In certain embodiments, the invention relates to the aforementioned method, wherein said at least one amino acid is leucine; and said at least one fluorinated amino acid is trifluoroisoleucine.

In certain embodiments, the invention relates to the aforementioned method, wherein said at least one amino acid is isoleucine; and said at least one fluorinated amino acid is trifluoroisoleucine.

In certain embodiments, the invention relates to the aforementioned method, wherein said at least one amino acid is alanine; and said at least one fluorinated amino acid is trifluoroisoleucine.

In certain embodiments, the invention relates to the aforementioned method, wherein said at least one amino acid is valine; and said at least one fluorinated amino acid is trifluoroisoleucine.

In certain embodiments, the invention relates to the aforementioned method, wherein said at least one amino acid is leucine; and said at least one fluorinated amino acid is trifluoronorvaline.

In certain embodiments, the invention relates to the aforementioned method, wherein said at least one amino acid is isoleucine; and said at least one fluorinated amino acid is trifluoronorvaline.

In certain embodiments, the invention relates to the aforementioned method, wherein said at least one amino acid is alanine; and said at least one fluorinated amino acid is trifluoronorvaline.

In certain embodiments, the invention relates to the aforementioned method, wherein said at least one amino acid is valine; and said at least one fluorinated amino acid is trifluoronorvaline.

In certain embodiments, the invention relates to the aforementioned method, wherein said at least one amino acid is glycine; and said at least one fluorinated amino acid is trifluoronorvaline.

In certain embodiments, the invention relates to the aforementioned method, wherein said at least one amino acid is glutamic acid; and said at least one fluorinated amino acid is trifluoronorvaline.

In certain embodiments, the invention relates to the aforementioned method, wherein said at least one amino acid is phenylalanine; and said at least one fluorinated amino acid is trifluoronorvaline.

In certain embodiments, the invention relates to the aforementioned method, wherein said at least one amino acid is leucine; and said at least one fluorinated amino acid is trifluoromethionine.

In certain embodiments, the invention relates to the aforementioned method, wherein said at least one amino acid is isoleucine; and said at least one fluorinated amino acid is trifluoromethionine.

In certain embodiments, the invention relates to the aforementioned method, wherein said at least one amino acid is alanine; and said at least one fluorinated amino acid is trifluoromethionine.

In certain embodiments, the invention relates to the aforementioned method, wherein said at least one amino acid is valine; and said at least one fluorinated amino acid is trifluoromethionine.

In certain embodiments, the invention relates to the aforementioned method, wherein said at least one amino acid is glycine; and said at least one fluorinated amino acid is trifluoromethionine.

In certain embodiments, the invention relates to the aforementioned method, wherein said at least one amino acid is glutamic acid; and said at least one fluorinated amino acid is trifluoromethionine.

In certain embodiments, the invention relates to the aforementioned method, wherein said at least one amino acid is phenylalanine; and said at least one fluorinated amino acid is trifluoromethionine.

In certain embodiments, the invention relates to the aforementioned method, wherein said at least one amino acid is leucine; and said at least one fluorinated amino acid is trifluoromethylmethionine.

In certain embodiments, the invention relates to the aforementioned method, wherein said at least one amino acid is isoleucine; and said at least one fluorinated amino acid is trifluoromethylmethionine.

In certain embodiments, the invention relates to the aforementioned method, wherein said at least one amino acid is alanine; and said at least one fluorinated amino acid is trifluoromethylmethionine.

In certain embodiments, the invention relates to the aforementioned method, wherein said at least one amino acid is valine; and said at least one fluorinated amino acid is trifluoromethylmethionine.

In certain embodiments, the invention relates to the aforementioned method, wherein said at least one amino acid is glycine; and said at least one fluorinated amino acid is trifluoromethylmethionine.

In certain embodiments, the invention relates to the aforementioned method, wherein said at least one amino acid is glutamic acid; and said at least one fluorinated amino acid is trifluoromethylmethionine.

In certain embodiments, the invention relates to the aforementioned method, wherein said at least one amino acid is phenylalanine; and said at least one fluorinated amino acid is trifluoromethylmethionine.

In certain embodiments, the invention relates to the aforementioned method, wherein said natural or non-natural polypeptide has the sequence GIGKFLHAAKKFAKAFVAEIMNS.

In certain embodiments, the invention relates to the aforementioned method, wherein said natural or non-natural polypeptide has the sequence RAGLQFPVGRVHRLLRK.

In certain embodiments, the invention relates to the aforementioned method, wherein said natural or non-natural polypeptide has the sequence TRSSRAGLQFPVGRVHRLLRK.

In certain embodiments, the invention relates to the aforementioned method, wherein said natural or non-natural polypeptide has the sequence QHWSYLLRP.

In certain embodiments, the invention relates to the aforementioned method, wherein said natural or non-natural polypeptide has the sequence KCNTATCATQRLANFLVHSSNNFGPILPPTNVGSNTY.

In certain embodiments, the invention relates to the aforementioned method, wherein said natural or non-natural polypeptide has the sequence HGEGTFTSDLSKQMEEEAVRXIEWLKNGGPSSGAPPPS.

In certain embodiments, the invention relates to the aforementioned method, wherein said natural or non-natural polypeptide has the sequence HAEGTFTSDVSSYLEGQAAKEFIAWLVKGR.

In certain embodiments, the invention relates to the aforementioned method, wherein said natural or non-natural polypeptide has the sequence SPKMVQGSGCFGRKMDRISSSSGLGCKVLRRK.

In certain embodiments, the invention relates to the aforementioned method, wherein said natural or non-natural polypeptide has the sequence YTSLIHSLIEESQNQQELNEQELLELDKWASLWNWF.

In certain embodiments, the invention relates to the aforementioned method, wherein said natural or non-natural polypeptide has the sequence VVYTDCTESGQNLCLCEGSNVCGQGNKCILGSDGEKNQCVTGEGTPKPQSHNDGD FEEIPEEYLQ.

In certain embodiments, the invention relates to the aforementioned method, wherein said natural or non-natural polypeptide has the sequence MPLWVFFFVILTLSNSSHCSPPPPLTLRMRRYADAIFTNSYRKVLGQLSARKLLQDI MSRQQGESNQERGARARLGRQVDSMWAEQKQMELESILVALLQKHSRNSQG.

In certain embodiments, the invention relates to the aforementioned method, wherein said natural or non-natural polypeptide has the sequence MKPIQKLLAGLILLTSCVEGCSSQHWSYGLRPGGKRDAENLIDSFQEIVKEVGQLAE TQRFECTTHQPRSPLRDLKGALESLIEEETGQKKI.

In certain embodiments, the invention relates to the aforementioned method, wherein said natural or non-natural polypeptide has the sequence MALWMRLLPLLALLALWGPDPAAAFVNQHLCGSHLVEALYLVCGERGFFYTPKT RREAEDLQVGQVELGGGPGAGSLQPLALEGSLQKRGIVEQCCTSICSLYQLENYCN.

### COMPOUNDS OF THE INVENTION

In certain embodiments, the invention relates to a polypeptide comprising at least one fluorinated amino acid wherein said polypeptide has a sequence selected from the group consisting of GIGKFXHAAKKFAKAFVAEXMNS; GIGKFXHAXKKFXKAFXAEXMNS; RAGLQFPVGRVHRXXRK; TRSSRAGLQFPVGRVHRXXRK; HXEGTFTSDVSSYLEGQAAKEFIAWLVKGR; HAXGTFTSDVSSYLEGQAAKEFIAWLVKGR; HXXGTFTSDVSSYLEGQAAKEFIAWLVKGR; HXXGTFTSDVSSYLEGQAAKEFIAWLVKGR; HXEGTFTSDVSSYLEGQAAKEXIAWLVKGR; HAXGTFTSDVSSYLEGQAAKEFXAWLVKGR; and HXEGTFTSDVSSYLEGQAAKEFIAWXVKGR; wherein X is a fluorinated amino acid.

In certain embodiments, the invention relates to the aforementioned polypeptide, wherein said polypeptide has the sequence GIGKFXHAAKKFAKAFVAEXMNS.

In certain embodiments, the invention relates to the aforementioned polypeptide, wherein said polypeptide has the sequence GIGKFXHAKFXKAFXAEXMNS.

In certain embodiments, the invention relates to the aforementioned polypeptide, wherein said polypeptide has the sequence RAGLQFPVGRVHRXXRK.

In certain embodiments, the invention relates to the aforementioned polypeptide, wherein said polypeptide has the sequence TRSSRAGLQFPVGRVHRXXRK.

In certain embodiments, the invention relates to the aforementioned polypeptide, wherein said polypeptide has a sequence selected from the group consisting of HXEGTFTSDVSSYLEGQAAKEFIAWLVKGR; HAXGTFTSDVSSYLEGQAAKEFIAWLVKGR; HXXGTFTSDVSSYLEGQAAKEFIAWLVKGR; HXXGTFTSDVSSYLEGQAAKEFIAWLVKGR; HXEGTFTSDVSSYLEGQAAKEXIAWLVKGR; HAXGTFTSDVSSYLEGQAAKEFXAWLVKGR; and HXEGTFTSDVSSYLEGQAAKEFIAWXVKGR.

In certain embodiments, the invention relates to the aforementioned polypeptide, wherein said polypeptide has the sequence HXEGTFTSDVSSYLEGQAAKEFIAWLVKGR.

In certain embodiments, the invention relates to the aforementioned polypeptide, wherein said polypeptide has the sequence HAXGTFTSDVSSYLEGQAAKEFIAWLVKGR.

In certain embodiments, the invention relates to the aforementioned polypeptide, wherein said polypeptide has the sequence HXXGTFTSDVSSYLEGQAAKEFIAWLVKGR.

In certain embodiments, the invention relates to the aforementioned polypeptide, wherein said polypeptide has the sequence HXXGTFTSDVSSYLEGQAAKEFIAWLVKGR.

In certain embodiments, the invention relates to the aforementioned polypeptide, wherein said polypeptide has the sequence HXEGTFTSDVSSYLEGQAAKEXIAWLVKGR.

In certain embodiments, the invention relates to the aforementioned polypeptide, wherein said polypeptide has the sequence HAXGTFTSDVSSYLEGQAAKEFXAWLVKGR.

In certain embodiments, the invention relates to the aforementioned polypeptide, wherein said polypeptide has the sequence HXEGTFTSDVSSYLEGQAAKEFIAWXVKGR.

In certain embodiments, the invention relates to the aforementioned polypeptide, wherein the fluorinated amino acid X is selected from the group consisting of trifluoroleucine, 4,4,4-trifluorovaline, 5,5,5-trifluoroleucine, trifluorovaline, hexafluorovaline, trifluoroisoleucine, trifluoronorvaline, hexafluoroleucine, 5,5,5,5′,5′,5′-hexafluoroleucine, trifluoromethionine, trifluoromethylmethionine and fluorophenylalanine.

In certain embodiments, the invention relates to a polypeptide, comprising at least one fluorinated amino acid replacement for at least one replaced natural amino acid, wherein said at least one fluorinated amino acid replacement is selected from the group consisting of trifluoroleucine, 4,4,4-trifluorovaline, 5,5,5-trifluoroleucine, trifluorovaline, hexafluorovaline, trifluoroisoleucine, trifluoronorvaline, hexafluoroleucine, 5,5,5,5′,5′,5′-hexafluoroleucine, trifluoromethionine, trifluoromethylmethionine and fluorophenylalanine; and said polypeptide is selected from the group consisting of: GIGKFLHAAKKFAKAFVAEIMNS, RAGLQFPVGRVHRLLRK, TRSSRAGLQFPVGRVHRLLRK, QHWSYLLRP, KCNTATCATQRLANFLVHSSNNFGPILPPTNVGSNTY, HGEGTFTSDLSKQMEEEAVRXIEWLKNGGPSSGAPPPS, HAEGTFTSDVSSYLEGQAAKEFIAWLVKGR, SPKMVQGSGCFGRKMDRISSSSGLGCKVLRRK, YTSLIHSLIEESQNQQELNEQELLELDKWASLWNWF, VVYTDCTESGQNLCLCEGSNVCGQGNKCILGSDGEKNQCVTGEGTPKPQSHNDGD FEEIPEEYLQ, MPLWVFFFVILTLSNSSHCSPPPPLTLRMRRYADAIFTNSYRKVLGQLSARKLLQDI MSRQQGESNQERGARARLGRQVDSMWAEQKQMELESILVALLQKHSRNSQ, MKPIQKLLAGLILLTSCVEGCSSQHWSYGLRPGGKRDAENLIDSFQEIVKEVGQLAE TQRFECTTHQPRSPLRDLKGALESLIEEETGQKKI, and MALWMRLLPLLALLALWGPDPAAAFVNQHLCGSHLVEALYLVCGERGFFYTPKT RREAEDLQVGQVELGGGPGAGSLQPLALEGSLQKRGIVEQCCTSICSLYQLENYCN.

In certain embodiments, the invention relates to the aforementioned polypeptide, wherein the at least one replaced natural amino acid is selected from the group consisting of leucine, isoleucine, valine and alanine.

In certain embodiments, the invention relates to the aforementioned polypeptide, wherein the at least one replaced natural amino acid is selected from the group consisting of leucine.

In certain embodiments, the invention relates to the aforementioned polypeptide, wherein said at least one fluorinated amino acid replacement is 5,5,5,5′,5′,5′-hexafluoroleucine.

In certain embodiments, the invention relates to the aforementioned polypeptide, wherein the at least one replaced natural amino acid is selected from the group consisting of leucine, isoleucine, valine and alanine; and said at least one fluorinated amino acid replacement is 5,5,5,5′,5′,5′-hexafluoroleucine.

In certain embodiments, the invention relates to the aforementioned polypeptide, wherein the at least one replaced natural amino acid is selected from the group consisting of leucine; and said at least one fluorinated amino acid replacement is 5,5,5,5′,5′,5′-hexafluoroleucine.

In certain embodiments, the invention relates to a polypeptide, comprising at least one fluorinated amino acid replacement, wherein said at least one fluorinated amino acid replacement is selected from the group consisting of trifluoroleucine, 5,5,5-trifluoroleucine, hexafluoroleucine, and 5,5,5,5′,5′,5′-hexafluoroleucine; each instance of X is independently leucine or a fluorinated amino acid replacement; and said polypeptide is selected from the group consisting of: GIGKFXHAAKKFAKAFVAEIMNS, RAGXQFPVGRVHRXXRK, TRSSRAGXQFPVGRVHRXXK, QHWSYXXRP, KCNTATCATQRXANFXVHSSNNFGPIXPPTNVGSNTY, HGEGTFTSDXSKQMEEEAVRXIEWXKNGGPSSGAPPPS, HAEGTFTSDVSSYXEGQAAKEFIAWXVKGR, SPKMVQGSGCFGRKMDRISSSSGXGCKVXRRK, YTSXIHSXIEESQNQQEXNEQEXXEXDKWASXWNWF, VVYTDCTESGQNXCXCEGSNVCGQGNKCIXGSDGEKNQCVTGEGTPKPQSHNDG DFEEIPEEYXQ, MPXWVFFFVIXTXSNSSHCSPPPPXTXRMRRYADAIFTNSYRKVXGQXSARKXXQ DIMSRQQGESNQERGARARXGRQVDSMWAEQKQMEXESIXVAXXQKHSRNSQG, MKPIQKXXAGXIXXTSCVEGCSSQHWSYGXRPGGKRDAENXIDSFQEIVKEVGQX AETQRFECTTHQPRSPXRDXKGAXESXIEEETGQKKI, and MAXWMRXXPXXAXWGPDPAAAFVNQHXCGSHXVEAXYXVCGERGFFYTP KTRREAEDXQVGQVEXGGGPGAGSXQPXAXEGSXQKRGIVEQCCTSICSXYQXEN YCN.

In certain embodiments, the invention relates to the aforementioned polypeptide, wherein said at least one fluorinated amino acid replacement is selected from the group consisting of 5,5,5,5′,5′,5′-hexafluoroleucine.

In certain embodiments, the invention relates to a polypeptide, comprising at least one fluorinated amino acid replacement for at least one replaced natural amino acid, wherein said at least one fluorinated amino acid replacement is selected from the group consisting of trifluoroleucine, 4,4,4-trifluorovaline, 5,5,5-trifluoroleucine, trifluorovaline, hexafluorovaline, trifluoroisoleucine, trifluoronorvaline, hexafluoroleucine, 5,5,5,5′,5′,5′-hexafluoroleucine, trifluoromethionine, trifluoromethylmethionine and fluorophenylalanine; each instance of X is independently a fluorinated amino acid replacement; and said polypeptide is selected from the group consisting of HXEGTFTSDVSSYLEGQAAKEFIAWLVKGR; HAXGTFTSDVSSYLEGQAAKEFIAWLVKGR; HXXGTFTSDVSSYLEGQAAKEFIAWLVKGR; HXXGTFTSDVSSYLEGQAAKEFIAWLVKGR; HXEGTFTSDVSSYLEGQAAKEXIAWLVKGR; HAXGTFTSDVSSYLEGQAAKEFXAWLVKGR; and HXEGTFTSDVSSYLEGQAAKEFIAWXVKGR.

In certain embodiments, the invention relates to the aforementioned polypeptide, wherein the at least one replaced natural amino acid is selected from the group consisting of leucine, isoleucine, alanine, glycine, glutamic acid, and phenylalanine.

In certain embodiments, the invention relates to the aforementioned polypeptide, wherein the at least one replaced natural amino acid is selected from the group consisting of leucine.

In certain embodiments, the invention relates to the aforementioned polypeptide, wherein said at least one fluorinated amino acid replacement is 5,5,5,5′,5′,5′-hexafluoroleucine.

In certain embodiments, the invention relates to the aforementioned polypeptide, wherein the at least one replaced natural amino acid is selected from the group consisting of leucine, isoleucine, alanine, glycine, glutamic acid, and phenylalanine; and said at least one fluorinated amino acid replacement is 5,5,5,5′,5′,5′-hexafluoroleucine.

In certain embodiments, the invention relates to the aforementioned polypeptide, wherein the at least one replaced natural amino acid is selected from the group consisting of leucine; and said at least one fluorinated amino acid replacement is 5,5,5,5′,5′,5′-hexafluoroleucine.

In certain embodiments, the invention relates to a polypeptide, comprising at least one fluorinated amino acid replacement, wherein said at least one fluorinated amino acid replacement is selected from the group consisting of trifluoroleucine, 5,5,5-trifluoroleucine, hexafluoroleucine, and 5,5,5,5′,5′,5′-hexafluoroleucine; each instance of X is independently leucine or a fluorinated amino acid replacement; and said polypeptide is selected from the group consisting of HXEGTFTSDVSSYLEGQAAKEFIAWLVKGR; HAXGTFTSDVSSYLEGQAAKEFIAWLVKGR; HXXGTFTSDVSSYLEGQAAKEFIAWLVKGR; HXXGTFTSDVSSYLEGQAAKEFIAWLVKGR; HXEGTFTSDVSSYLEGQAAKEXIAWLVKGR; HAXGTFTSDVSSYLEGQAAKEFXAWLVKGR; and HXEGTFTSDVSSYLEGQAAKEFIAWXVKGR.

In certain embodiments, the invention relates to the aforementioned polypeptide, wherein said at least one fluorinated amino acid replacement is selected from the group consisting of 5,5,5,5′,5′,5′-hexafluoroleucine.

In certain embodiments, the invention relates to a polypeptide comprising at least one radiolabeled amino acid wherein said polypeptide has the sequence DLSK*QMEEEAVRLFIEWLKNGGPSSGAPPPS; wherein K* is a radiolabeled amino acid.

### Exemplification

The invention now being generally described, it will be more readily understood by reference to the following examples, which are included merely for purposes of illustration of certain aspects and embodiments of the present invention, and are not intended to limit the invention.

### Example 1

### Synthesis of Bis-trifluoromethyl olefin (2)

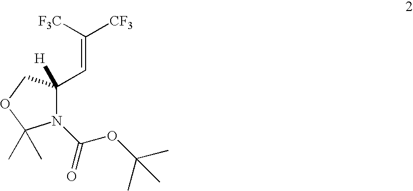

Typical procedure for the coupling reaction: To a stirred solution of the Garner aldehyde 1 (7.0 g, 31.0 mmol) and PPh₃ (57 g, 217 mmol) in dry Et₂O (300 mL) was added 2,2,4,4-tetrakis-(trifluoromethyl)-1,3-dithietane (39.5 g, 108.5 mmol) at −78° C. under argon. The mixture was stirred for 3 d while being slowly warmed to room temperature. The reaction slowly accumulated an insoluble white solid which was filtered and the filtrate concentrated. The residue was further dissolved in n-pentane (300 mL) and filtered again to remove insoluble impurities. After removal of the solvent, the residue was subjected to flash column chromatography using n-pentane/Et₂O (6/1) as eluant to give pure 2 as a pale yellow oil (10.4 g, 92%). ¹H NMR (300 MHz, CDCl₃) δ 6.70 (d, 1H, J=8.7 Hz), 4.81 (bs, 1H), 4.23 (dd, 1H, J=6.9 Hz, 9.3 Hz), 3.79 (dd, 1H, J=3.9 Hz, 9.3 Hz), 1.65 (s, 3H), 1.56 (s, 3H), 1.42 (s, 9H); ¹⁹F NMR (282.6 MHz, CDCl₃/CFCl₃) δ −65.01 (d, 3F, J=5.9 Hz), −58.44 (d, 3F, J=5.9 Hz); FT-IR (film, νₘₐₓ, cm⁻) 2983m, 2935m, 2885w, 1713s, 1479w, 1460w, 1379s, 1230s, 1165s, 1110m, 971m; [α]_{D}^{26.1}+12.3° (c 1.7, CHCl₂); GC-MS (CI, CH₄): 364 (1, [M+1]⁺), 336 (18), 308 (100), 288 (98), 264 (37), 102 (2), 57 (9).

### Example 2

### Synthesis of Oxazolidine (3)

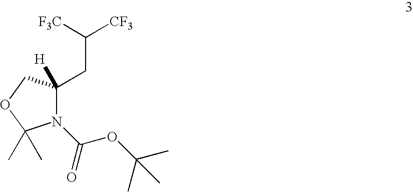

A 500 mL round bottomed flask was charged with a solution of 2 (10.3 g, 28.3 mmol) in THF (250 mL) and 10% Pd/C (40 g). The reaction flask was purged with argon and hydrogen sequentially and stirred under hydrogen at room temperature until uptake of H₂ ceased (24 hours). The catalyst was then separated from the reaction mixture by filtration (and can be used again). The filtrate was dried over anhydrous MgSO₄ and concentrated by rotary evaporation to give 3 (10.1 g, 98% yield) as a pale yellow oil. ¹H NMR (300 MH, CDCl₃) δ 4.23 (4.05) (m, 1H), 4.00 (dd, 1H, J=5.4 Hz, 9.3 Hz), 3.73 (d, 1H, J=9.3 Hz), 3.58 (3.05) (m, 1H), 2.18 (2.01) (m, 2H), 1.62 (1.58) (s, 3H), 1.48 (br. s, 12H); ¹³C NMR (75.5 MHz, CDCl₃) δ 153.22 (151.51) (C═O), 123.89 (q, 2×CF₃, ¹J_{CF}=284.0), 94.47 (94.03) (C), 80.85 (80.73) (C), 67.26 (66.65) (CH₂), 55.58 (55.12) (CH), 45.44 (45.12) (quintet, CH, ²J_{CF}=27.2 Hz), 28.98 (28.00) (CH₂), 28.25 (3×CH₃), 27.58 (26.90) (CH₃), 24.15 (22.86) (CH₃); ¹⁹F NMR (282.6 MHz, CDCl₃/CFCl₃) δ −67.68-−68.42 (m); FT-IR (film, νₘₐₓ, cm⁻¹): 2984m, 2941m, 2884w, 1704s, 1457m, 1393s, 1258s, 1168s, 1104s, 847m; [α]_{D}^{22.4}=+17.5° (c 0.4, CHCl₃); GC-MS (CI, CH₄): 366 (4, [M+1]⁺), 338 (16), 310 (100), 290 (48), 266 (48), 57 (8).

### Example 3

### Synthesis of N-Boc-5,5,5,5′,5′,5′-(S)-Hexafluoroleucinol (4)

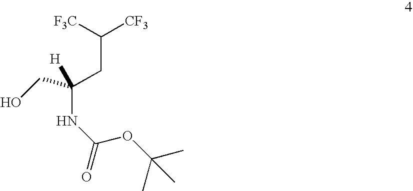

To a solution of 3 (10.1 g, 27.6 mmol) in CH₂Cl₂ (30 mL) was added 10 mL of trifluoroacetic acid (TFA). The reaction mixture was stirred at room temperature for 5 min. After removal of the solvent and TFA, the residue was partitioned between 150 mL of ethyl ether and 100 mL of H₂O. The organic layer was washed with water (20 mL×4), dried over MgSO₄, and concentrated to give 4 (7.2 g, 80% yield) as a white solid. The aqueous layers contain a completely deprotected product due to cleavage of the BOC moiety as evidenced by ninhydrin active material. This hexafluoroamino alcohol can be converted back to 4 by protecting the free amine group as a BOC amide. ¹H NMR (300 MHz, CDCl₃) δ5.03 (d, 1H, J=8.1 Hz), 3.84 (m, 1H), 3.70 (m, 2H), 3.20 (m, 1H), 3.10 (br. s, 1H), 1.98 (m, 2H), 1.45 (s, 9H); ¹³C NMR (75.5 MHz, CDCl₃) δ 156.57 (C═O), 124.00 (q, 2×CF₃, ¹J_{CF}=284.0 Hz), 80.58 (C), 66.08 (CH₂), 50.57 (CH), 45.09 (m, CH, ²J_{CF}=28.1 Hz), 28.38 (3×CH₃), 26.44 (CH₂); ¹⁹F NMR (282.6 MHz, CDCl₃/CFCl₃) δ −67.96 (m), −68.46 (m); FT-IR (KBr pellet, νₘₐₓ, cm⁻¹) 3397s (br), 3253s, 3068m, 2981s, 2948m, 1686s, 1552s, 1369s, 1289s, 1174s, 1145s, 1055s; [α]_{D}^{22.9}=−14.4° (c 1.0, CH₃OH); GC-MS (CI, CH₄): 326 (8, [M+1]⁺), 298 (14), 270 (100), 226 (20), 57 (2); m.p.=114-115° C.

### Example 4

### Synthesis of N-Boc-5,5,5,5′,5′,5′-(S)-Hexafluoroleucine (5)

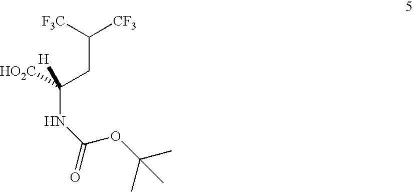

A mixture of 4 (7.1 g, 21.8 mmol) and pyridinium dichromate (33 g, 88 mmol) in DMF (150 mL) was stirred under argon at room temperature for 24 hrs. before 150 mL of H₂O was added. The mixture was then extracted with ethyl ether (400 mL×2). The combined ether layers were washed with 1 N HCl (80 mL×2) and concentrated until about 150 mL of solution left. This solution was washed with 5% NaHCO₃ (150 mL×3). The combined aqueous layers were acidified to pH 2 with 3 N HCl, extracted with ether again (400 mL×2). The ether layers were then dried over MgSO₄ and concentrated to give 5 (5.2 g, 70%) as a white solid. ¹H NMR (300 MHz, CDCl₃) δ 7.36 (5.21) (d, 1H, J=6.3 Hz), 4.41 (m, 1H), 3.37 (m, 1H), 2.43-2.11 (br. m, 2H), 1.47 (s, 9H); ¹⁹F NMR (282.6 MHz, CDCl₃/CFCl₃) δ −67.87-−68.23 (m); FT-IR (KBr pellet, νₘₐₓ, cm⁻¹) 3358-2500m (br.), 3245s, 3107m, 2989s, 2980m, 1725s, 1712s, 1657s, 1477s, 1458s, 1404s, 1296s, 1277s, 1258s, 916m; [α]_{D}^{21.8}=−23.0° (c 1.0, CH₃OH); GC-MS (CI, CH₄): 3 40 (21, [M+1]⁺), 312 (7), 284 (100), 264 (16), 240 (19), 57 (39); m.p.=85-91° C.

### Example 5

### Synthesis of 5,5,5,5′,5′,5′-(S)-Hexafluoroleucine (6)

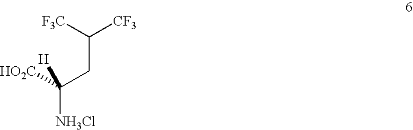

A solution of 5 (581 mg, 1.7 mmol) in 5 mL of TFA/CH₂Cl₂ (⅔) was stirred for 30 min. After removal of the solvents, the residue was partitioned between 1 N HCl (10 mL×3) and ethyl ether (10 mL). The combined aqueous layers were freeze dried to give 6 (446 mg, 95% yield) as a white solid.

### Example 6

### Synthesis of Dipeptide (8)

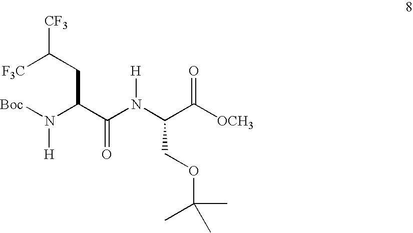

To a stirred solution of 5 (11 mg, 0.03 mmol) in anhydrous DMF (1 mL) was added diisopropyl ethyl amine (13 mg, 0.1 mmol), HBTU (13 mg, 0.03 mmol), and H-Ser(t-Bu)-OMe.HCl (14 mg, 0.065 mmol) sequentially. The mixture was stirred at room temperature for 40 min before 6 mL of H₂O was added. The reaction mixture was extracted with ether (15 ml) and the organic layer was further washed with 1 N HCl (5 mL×2) and 5% NaHCO₃ solution (5 ml), dried over MgSO₄, and concentrated to afford 8 (13 mg, 87% yield) as a white solid. ¹H NMR (300 MHz, CDCl₃) ε 6.68 (d, 1H, J=8.1 Hz), 5.21 (d, 1H, J=8.1 Hz), 4.64 (m, 1H), 4.40 (m, 1H), 3.86 (dd, 1H, J=2.7 Hz, 9.3 Hz), 3.76 (s, 3H), 3.56 (dd, 1H, J=3.3 Hz, 9.3 Hz), 3.50 (m, 1H), 2.33-2.10 (br. m, 2H), 1.45 (s, 9H), 1.14 (s, 9H).

### Example 7

### N-Boc-4,4,4-trifluorovalinol (2)

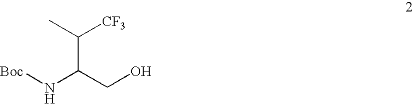

To a suspension of Boc-DL-trifluorovaline (1.30 g, 4.79 mmol) and NaHCO₃ (1.21 g, 14.37 mmol) in 20 mL of dry DMF was added 0.33 mL of CH₃I (5.27 mmol) at room temperature under argon. The resulting mixture was stirred for 5 h and then partitioned between 75 mL of ethyl acetate and 50 mL of water. The organic layer was washed with water (3×50 mL), dried over MgSO₄, and concentrated to yield 1.36 g (95%) of the Boc-DL-trifluorovaline methyl ester as a pale-yellow oil.

The Boc-TFV methyl ester (855 mg, 3 mmol) was dissolved in 20 mL of methanol, and NaBH₄ (681 mg, 18 mmol) was added in small portions at 0° C. The reaction mixture was stirred overnight at room temperature and then diluted with 80 mL of ethyl acetate, washed with water (3×50 mL), and dried over MgSO₄. After removal of the solvent, the crude product (Boc-trifluorovalinol) was chromatographed on a silica gel column (silica gel, 300 g) using n-pentane/Et₂O (1:1) as eluant to give 452 mg of 2a as a pale-yellow solid (58%) and 214 mg of 2b as a white solid (28%).

### (2S,3R)-, (2R,3S)-N-Boc-4,4,4-trifluorovalinol (2a)

¹H NMR (300 MHz, CDCl₃) δ 5.04 (d, 1H, J=9.3 Hz), 4.02 (m, 1H), 3.62 (m, 3H), 2.61 (m, 1H), 1.44 (s, 9H), 1.15 (d, 3H, J=7.2 Hz); ¹³C NMR (75.5 MHz, CDCl₃) δ 156.20 (C═O), 127.83 (q, CF₃, ¹J_{CF}=279.9 Hz), 80.26 (C), 62.78 (CH₂), 51.09 (CH), 38.47 (q, CH, ²J_{CF}=25.6 Hz), 28.40 (3×CH₃), 8.76 (CH₃); ¹⁹F NMR (282.6 MHz, CDCl₃/CFCl₃) δ −70.63 (d, 3F, J=9.0 Hz); FT-IR (KBr pellet, νₘₐₓ, cm⁻¹) 3435s, 3300s, 2990s, 2979m, 2954m, 1691s, 1539s, 1537s, 1265s, 1172s, 1125; GC-MS (CI, CH₄): 258 (14, [M+1]⁺), 242 (4), 202 (100), 158 (37), 57 (14).

### (2S,3S)-, (2R,3R)-N-Boc-4,4,4-trifluorovalinol (2b)

¹H NMR (300 MHz, CDCl₃) δ 5.11 (d, 1H, J=8.4 Hz), 3.80 (m, 1H), 3.66 (m, 2H), 3.45 (t, 1H, J=5.7 Hz), 2.53 (m, 1H), 1.42 (s, 9H), 1.15 (d, 3H, J=7.2 Hz); ¹³C NMR (75.5 MHz, CDCl₃) δ 156.43 (C═O), 127.91 (q, CF₃, ¹J_{CF}=280.2 Hz), 80.30 (C), 62.92 (CH₂), 52.56 (CH), 38.89 (q, CH, ²J_{CF}=24.8 Hz), 28.40 (3×CH₃), 10.59 (CH₃); ¹⁹F NMR (282.6 MHz, CDCl₃/CFCl₃) δ 68.76 (d, 3F, J=8.5 Hz); FT-IR (film, vₘₐₓ, cm⁻¹): 3436s, 3302s, 3012m, 2990m, 2954m, 1691s, 1532s, 1265s, 1172s, 1127s; GC-MS (CI, CH₄): 258 (14, [M+1]⁺), 242 (4), 202 (100), 182 (8), 57 (14).

### Example 8

### (2S,3R)-, (2R,3S)-N-Ac-4,4,4-trifluorovaline (3a)

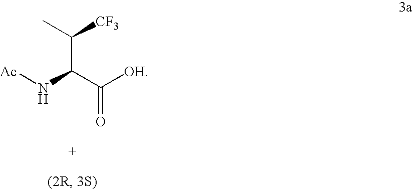

A solution of alcohol 2a (257 mg, 1 mmol) in 4 mL of dry DMF was treated with PDC (2.26 g, 6 mmol) at room temperature under argon and stirred overnight. The reaction mixture was then diluted with 20 mL of diethyl ether/30 mL of saturated NaHCO₃ solution. The organic layer was washed with 10 mL of saturated NaHCO₃. The combined aqueous layers were acidified to pH 2 with 3 N HCl and extracted with diethyl ether (2×50 mL). The combined organic layers were dried over MgSO₄ and concentrated to yield 176 mg of the corresponding Boc-trifluorovaline (65%).

Boc-TFV (176 mg, 0.65 mmol) was treated with 4 mL of 40% trifluoroacetic acid in CH₂Cl₂ for 10 min. After removal of the solvent, the residue was dissolved in 2 mL of water, treated with NaOH (260 mg, 6.5 mmol) at 0° C., followed by dropwise addition of acetic anhydride (0.13 mL, 1.3 mmol). The reaction mixture was stirred at 0° C. for 30 min before it was allowed to warm to room temperature. After stirring for another 1.5 h, the mixture was diluted with 10 mL of water, acidified to pH 2 with 1 N HCl, and extracted with ethyl acetate (2×60 mL). The combined organic layers were dried over MgSO₄ and concentrated to give the desired product 3a as a white solid (132 mg, 95%). ¹H NMR (300 MHz, D₂O) δ 4.96 (d, 1H, J=3.0 Hz), 3.07 (m, 1H), 2.04 (s, 3H), 1.15 (d, 3H, J=7.2 Hz); ¹⁹F NMR (282.6 MHz, D₂O/CF₃CO₂H) δ −71.63 (d, 3F, J=8.8 Hz); FT-IR (KBr pellet, νₘₐₓ, cm⁻¹) 3397s (br), 3253s, 3068m, 2981s, 2948m, 1686s, 1552s, 1369s, 1289s, 1174s, 1145s, 1055s; GC-MS (CI, CH₄): 214 (100, [M+1]⁺), 196 (9), 172 (33), 82 (33), 57 (6).

### (2S,3S)-, (2R,3R)-N-Ac-4,4,4-trifluorovaline (3b)

¹H NMR (300 MHz, D₂O) δ 4.67 (d, 1H, J=3.3 Hz), 3.07 (m, 1H), 2.04 (s, 3H), 1.17 (d, 3H, J=7.2 Hz); ¹⁹F NMR (282.6 MHz, D₂O/CF₃CO₂H) δ −69.43 (d, 3F, J=8.8 Hz); FT-IR (KBr pellet, νₘₐₓ, cm⁻¹) 3397s (br), 3253s, 3068m, 2981s, 2948m, 1686s, 1552s, 1369s, 1289s, 1174s, 1145s, 1055s; GC-MS (CI, CH₄): 214 (100, [M+1]⁺), 196 (9), 172 (33), 101 (10), 82 (33), 57 (6).

### Example 9

### (2S,3R)-4,4,4-Trifluorovaline (4a)

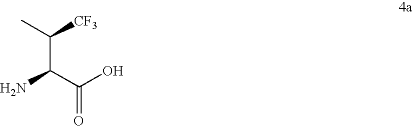

To a solution of 3a (107 mg, 0.5 mmol) in 1 mL of pH 7.9 aq. LiOH/HOAc was added porcine kidney acylase I (10 mg) at 25° C. The mixture was stirred at 25° C. for 48 h (pH was maintained at 7.5 by periodic addition of 1 N LiOH). The reaction was then diluted with 5 mL of water, acidified to pH 5.0, heated to 60° C. with Norit, and filtered. The filtrate was acidified to pH 1.5 and extracted with ethyl acetate (2×10 mL). The aqueous layer was freeze-dried to give 49 mg of 4a (95%). The combined organic layers were concentrated, and the residue refluxed in 3 N HCl for 6 h, then freeze-dried to yield 50 mg of 4c (98%).

The other two diastereomers, 4b and 4d, were obtained from 3b using the same procedure.

### (2S,3R)-4,4,4-Trifluorovaline (4a)

¹H NMR (300 MHz, D₂O) δ 4.24(dd; 1H, J=2.1, 3.9Hz), 3.23 (m, 1H), 1.30 (d, 3H, J=7.2 Hz); ¹⁹F NMR (282.6 MHz, D₂O/CF₃CO₂H) δ −71.69 (d, 3F, J=9.3 Hz); [α]_{D}^{23.7}=+7.2° (c 0.75, 1 N HCl).

### (2S,3S)-4,4,4-Trifluorovaline (4b)

¹H NMR (300 MHz, D₂O) δ 4.35 (t, 1H, J=2.7 Hz), 3.27 (m, 1H), 1.22 (d, 3H, J=7.5 Hz); ¹⁹F NMR (282.6 MHz, D₂O/CF₃CO₂H) δ −70.04 (d, 3F, J=9.0 Hz); [α]_{D}^{23.3}=+12.8° (c 0.5, 1 N HCl).

### (2R,3S)-4,4,4-Trifluorovaline (4c)

¹H NMR (300 MHz, D₂O) δ 4.24 (dd, 1H, J=2.1, 3.9 Hz), 3.23 (m, 1H), 1.30 (d, 3H, J=7.2 Hz); ¹⁹F NMR (282.6 MHz, D₂O/CF₃CO₂H) δ −70.04 (d, 3F, J=9.0 Hz).

### (2R,3R)-4,4,4-Trifluorovaline (4d)

¹H NMR (300 MHz, D₂O) δ 4.35 (t, 1H, J=2.7 Hz), 3.27 (m, 1H), 1.22 (d, 3H, J=7.5 Hz); ¹⁹F NMR (282.6 MHz, D₂O/CF₃CO₂H) δ −71.69 (d, 3F, J=9.3 Hz).

### Example 10

### N-Boc-5,5,5-trifluoroleucine methyl ester (6)

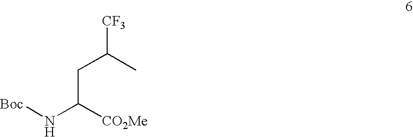

A mixture of Boc-DL-trifluoroleucine (1.25 g, 4.38 mmol), iodomethane (0.3 mL, 4.82 mmol), NaHCO₃ (1.1 g, 13.15 mmol), and dry DMF (20 mL) was stirred at room temperature under argon for 6 h, then diluted with 200 mL of ethyl acetate, and washed with water (4×100 mL). The organic layer was dried over Na₂SO₄ and concentrated to give 1.25 g of product as a pale-yellow oil (95%). Column chromatography on silica gel (500 g) using Et₂O/n-pentane (1:4) as eluant afforded 420 mg of (2S,4R)-, (2R,4S)-N-Boc-5,5,5-trifluoroleucine methyl ester (6a) (32%), 347 mg of (2S,4S)-, (2R,4R)-N-Boc-5,5,5-trifluoroleucine methyl ester (6b) (27%), and 337 mg of the mixture of 6a and 6b (26%).

### (2S,4R)-, (2R,4S)-N-Boc-5,5,5-trifluoroleucine methyl ester (6a)

¹H NMR (300 MHz, CDCl₃) δ 5.29 (d, 1H, J=6.9 Hz), 4.32 (m, 1H), 3.70 (s, 3H), 2.31 (m, 1H), 2.12 (m, 1H), 1.58 (m, 1H), 1.37 (s, 9H), 1.11 (d, 3H, J=6.9 Hz); ¹³C NMR (75.5 MHz, CDCl₃) δ 172.72 (C═O), 155.29 (C═O), 128.09 (q, CF₃, ¹J_{CF}=278.9 Hz), 80.27 (C), 52.54 (CH₃), 51.70 (CH), 35.13 (q, CH, ²J_{CF}=26.4 Hz), 32.98 (CH₂), 28.30 (3×CH₃), 13.17 (CH₃); ¹⁹F NMR (282.6 MHz, CDCl₃/CFCl₃) δ −74.15 (d, 3F, J=8.2 Hz); FT-IR (film, νₘₐₓ, cm⁻¹) 3360m, 2984m, 2938m, 1747s, 1716s, 1520s, 1368s, 1269s, 1168s, 1133m; GC-MS (CI, CH₄): 300 (2, [M+1]⁺), 284 (7), 244 (100), 200 (66), 82 (21), 57 (24).

### (2S,4S)-, (2R,4R)-N-Boc-5,5,5-trifluoroleucine methyl ester (6b)

¹H NMR (300 MHz, CDCl₃) δ 5.02 (d, 1H, J=8.7 Hz), 4.38 (m, 1H), 3.76 (s, 3H), 2.32 (m, 1H), 1.91-1.74 (br. m, 2H), 1.44 (s, 9H), 1.20 (d, 3H, J=6.9 Hz); ¹³C NMR (75.7 MHz, CDCl₃) δ 173.03 (C═O), 155.86 (C═O), 128.24 (q, CF₃, ¹J_{CF}=278.9 Hz), 80.57 (C), 52.80 (CH₃), 50.83 (CH), 35.02 (q, CH, ²J_{CF}=26.9 Hz), 33.00 (CH₂), 28.42 (3×CH₃), 12.28 (CH₃); ¹⁹F NMR (282.6 MHz, CDCl₃/CFCl₃) δ −74.03 (d, 3F, J=8.7 Hz); FT-IR (KBr pellet, νₘₐₓ, cm⁻¹) 3368s, 3014m, 2983s, 2961m, 1763s, 1686s, 1527s, 1265s, 1214s, 1170s, 1053s, 1028s; GC-MS (CI, CH₄): 300 (2, [M+1]⁺), 284 (7), 244 (100), 224 (30), 200 (66), 57 (24).

### Example 11

### (2S,4R)-, (2R,4S)-N-Ac-5,5,5-trifluoroleucine (7a)

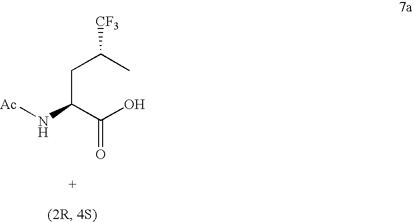

### (2S,4R)-, (2R,4S)-N-Boc-5,5,5-trifluoroleucinol

To a solution of 6a (420 mg, 1.4 mmol) in methanol (10 mL) was added NaBH₄ (531 mg, 14.0 mmol) in small portions. The reaction mixture was stirred at room temperature for 1 h before removal of the solvent. The residue was partitioned between 100 mL of ethyl acetate and 50 mL of water. The aqueous layer was extracted with 100 mL of ethyl acetate. The combined organic layers were dried over Na₂SO₄ and concentrated to yield 357 mg of the desired product as a white solid (94%). ¹H NMR (300 MHz, CDCl₃) δ 4.74 (m, 1H), 3.71 (m, 2H), 3.58 (m, 1H), 2.31 (m, 1H), 2.14 (m, 1H), 1.92 (m, 1H), 1.45 (s, 9H), 1.17 (d, 3H, J=7.0 Hz). ¹³C NMR (75.5 MHz, CDCl₃) δ 156.26 (C═O), 128.41 (q, CF₃, ¹J_{CF}=279.4 Hz), 80.14 (C), 64.78 (CH₂), 50.73 (CH), 35.59 (q, CH, ²J_{CF}=29.6 Hz), 31.74 (CH₂), 28.52 (3×CH₃), 13.71 (CH₃); ¹⁹F NMR (282.6 MHz, CDCl₃/CFCl₃) δ −73.84 (br. s, 3F); GC-MS (CI, CH₄): 272 (100, [M+1]⁺), 216 (68), 172 (26), 57 (11).

### (2S,4S)-, (2R,4R)-N-Boc-5,5,5-trifluoroleucinol

¹H NMR (300 MHz, CDCl₃) δ 4.58 (m, 1H), 3.79 (m, 1H), 3.68 (m, 1H), 3.58 (m, 1H), 2.27 (m, 1H), 2.05 (m, 1H), 1.80 (m, 1H), 1.45 (s, 9H), 1.18 (d, 3H, J=6.6 Hz). ¹³C NMR (75.5 MHz, CDCl₃) δ 156.47 (C═O), 128.56 (q, CF₃, ¹J_{CF}=278.7 Hz), 80.20 (C), 66.31 (CH₂), 49.49 (CH), 35.15 (q, CH, ²J_{CF}=26.7 Hz), 31.71 (CH₂), 28.50 (3×CH₃), 12.56 (CH₃); ¹⁹F NMR (282.6 MHz, CDCl₃/CFCl₃) δ −73.98 (d, 3F, J=8.5 Hz); GC-MS (CI, CH₄): 272 (100, [M+1]⁺), 172 (26), 57 (11).

### (2S,4R)-, (2R,4S) -N-Ac-5,5,5-trifluoroleucine (7a)

A mixture of (2S,4R)-, (2R,4S)-N-Boc-5,5,5-trifluoroleucinol (330 mg, 1.23 mmol), PDC (4.62 g, 12.3 mmol), and dry DMF (2.5 mL) was stirred at room temperature under argon for 4 h, then diluted with 50 mL of ethyl acetate and 50 mL of water. The organic layer was washed with 30 mL of 1N HCl and 2×30 mL of water, dried over MgSO₄, and concentrated to give 198 mg of (2S,4R)-, (2R,4S)-N-Boc-5,5,5-trifluoroleucine as a pale-brownish oil (60%).

A solution of the above product (180 mg, 0.63 mmol) in 2 mL of CH₂Cl₂ was treated with 0.5 mL of trifluoroacetic acid for 30 min at room temperature. After removal of the solvent, the yellowish residue was dissolved in 2 mL of water, treated with NaOH (126 mg, 3.15) at 0° C., and acetic anhydride (0.12 mL, 1.26 mmol) was then added dropwise. The reaction mixture was stirred at 0° C. for 30 min, then allowed to warm to room temperature. After stirring for another 1 h, the mixture was diluted with 30 mL of water, acidified to pH 2 with 3 N HCl, and extracted with ethyl acetate (2×90 mL). The combined organic layers were dried over Na₂SO₄ and concentrated to yield 136 mg of 7a as a white solid (95%). ¹H NMR (300 MHz, D₂O) δ 4.48 (dd, 1H, J=6.1, 8.8 Hz), 2.51 (m, 1H), 2.27 (m, 1H), 2.06 (s, 3H), 1.79 (m, 1H), 1.18 (d, 3H, J=7.0 Hz); ¹³C NMR (75.5 MHz, D₂O) δ 175.48 (C═O), 174.60 (C═O), 128.53 (q, CF₃, ¹J_{CF}=278.9 Hz), 51.24 (CH), 34.88 (q, CH, ²J_{CF}=26.6 Hz), 31.21 (CH₂), 21.90 (CH₃), 13.03 (CH₃); ¹⁹F NMR (282.8 MHz, D₂O/CF₃CO₂H) δ −73.68 (d, 3F, J=9.0 Hz); FT-IR (K1Br pellet, νₘₐₓ, cm⁻¹) 3343s, 3063-2487m (br.), 2932m, 2894m, 1709s, 1613s, 1549s, 1266s, 1179s, 1137s; GC-MS (CI, CH₄): 228 (100, [M+1]⁺), 211 (47), 186 (26), 140 (16), 57 (11).

### (2S,4S)-, (2R,4R)-N-Ac-5,5,5-trifluoroleucine (7b)

¹H NMR (300 MHz, D₂O) δ 4.48 (dd, 1H, J=3.8, 11.6 Hz), 2.41 (m, 1H), 2.07 (s, 3H), 2.15-1.91 (br. m, 2H), 1.16 (d, 3H, J=6.9 Hz); ¹³C NMR (75.5 MHz, D₂O) δ 178.35 (C═O), 177.38 (C═O), 131.09 (q, CF₃, ¹J_{CF}=278.3 Hz), 52.72 (CH), 37.31 (q, CH, ²J_{CF}=26.6 Hz), 33.06 (CH₂), 24.50 (CH₃), 13.90 (CH₃); ¹⁹F NMR (282.6 MHz, D₂O/CF₃CO₂H) δ −73.87 (d, 3F, J=8.5 Hz); FT-IR (KBr pellet, νₘₐₓ, cm⁻¹) 3336s, 2977m, 2949m, 2897m, 2615m, 2473s, 1711s, 1628s, 1551s, 1276s, 1250s, 1127s, 1095s; GC-MS (CI, CH₄): 228 (100, [M+1]⁺), 211 (47), 186 (26), 140 (16), 120 (3), 57 (11).

### Example 12

### (2S,4R)-5,5,5-Trifluoroleucine (8a)

To a solution of 7a (136 mg, 0.6 mmol) in 2 mL of pH 7.9 aqueous LiOH/HOAc was added porcine kidney acylase I (18 mg) at 27° C. The mixture was stirred at 27° C. for 48 h (pH was maintained at 7.5 by periodic addition of 1 N LiOH). It was further diluted with 5 mL of water, acidified to pH 5.0, heated to 60° C. with Norit, and filtered. The filtrate was acidified to pH 1.5 and extracted with ethyl acetate (2×50 mL). The aqueous layer was freeze-dried to give 63 mg of 8a (95%). The combined organic layers were concentrated, and the residue refluxed in 3 N HCl for 6 h, then freeze-dried to yield 64 mg of 8c (96%).

The other two diastereomers, 8b and 8d, were obtained from 7b using the same procedure.

### (2S,4R)-5, 5, 5-Trifluoroleucine (8a)

¹⁹F NMR (282.6 MHz, D₂O/CF₃CO₂H) δ −74.33 (d, 3F, J=9.0 Hz); [α]_{D}^{22.9}=+21.6° (c 0.5, 1N HCl).

### (2S,4S)-5,5,5-Trifluoroleucine (8b)

¹⁹F NMR (282.6 MHz, D₂O/CF₃CO₂H) δ −74.11 (d, 3F, J=8.2 Hz); [α]_{D}^{23.6}=−4.0° (c 0.8, 1N HCl).

### (2R,4S)-5,5,5-Trifluoroleucine (8c)

¹⁹F NMR (282.6 MHz, D₂O/CF₃CO₂H) δ −74.33 (d, 3F, J=9.0 Hz).

### (2R,4R)-5,5,5-Trifluoroleucine (8d)

¹⁹F NMR (282.6 MHz, D₂O/CF₃CO₂H) δ −74.11 (d, 3F, J=8.2 Hz).

### Example 13

### Boc-TFV(2S,3S)-Ser(Ot-Bu)-OMe(2S)

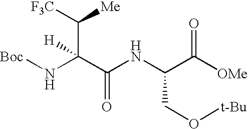

To a stirred solution of (2S,4S)-5,5,5-Trifluorovaline (4b) (5 mg, 0.02 mmol) in DMF (1 mL) was added diisopropylethyl amine (DIEA, 0.01 mL, 0.06 mmol), O-(benzotriazol-1-yl)-N,N,N′,N′-tetramethyluronium hexafluorophosphate (HBTU, 8 mg, 0.02 mmol), and the HCl salt of (2S)-H-Ser(Ot-Bu)-OMe (9 mg, 0.04 mmol), sequentially. The mixture was stirred at room temperature for 20 min before dilution with water (5 mL) and extraction with diethyl ether (15 mL). The organic layer was washed with 1 N HCl (2×5 mL) and 5% NaHCO₃ (2×8 mL), dried over MgSO₄, and concentrated to give 7 mg of the dipeptide (88%). ¹H NMR (300 MHz, CDCl₃) δ 6.92 (d, 1H, J=7.8 Hz), 5.16 (d, 1H, J=8.7 Hz), 4.65 (m, 1H), 4.39 (dd, 1H, J=5.1, 8.8 Hz), 3.81 (dd, 1H, J=2.7, 9.0 Hz), 3.74 (s, 3H), 3.56 (dd, 1H, J=3.0, 9.0 Hz), 3.04 (m, 1H), 1.46 (s, 9H), 1.23 (d, 3H, J=7.2 Hz), 1.14 (s, 9H); ¹⁹F NMR (282.6 MHz, CDCl₃/CFCl₃) δ −8.57 (d, 3F, J=8.7 Hz).

### Boc-TFV(2S,3R)-Ser(Ot-Bu)-OMe(2S)

¹⁹F NMR (282.6 MHz, CDCl₃/CFCl₃) δ −71.36 (d, 3F, J=7.9 Hz).

### Boc-TFV(2R,3S)-Ser(Ot-Bu)-OMe(2S)

¹⁹F NMR (282.6 MHz, CDCl₃/CFCl₃) δ −71.48 (d, 3F, J=8.5 Hz).

### Boc-TFV(2R,3R)-Ser(Ot-Bu)-OMe(2S)

¹⁹F NMR (282.6 MHz, CDCl₃/CFCl₃) δ −68.49 (d, 3F, J=9.0 Hz).

### Example 14

### Peptide Synthesis

Peptides were synthesized manually using the in-situ neutralization protocol² for t-Boc chemistry on a 0.075 mmol scale. MBHA and Boc-lys(2-Cl-Z)-Merrifield resins were used for peptides M2 (SEQ ID NO 1), M2F2 and M2F5 and peptides BII1 (SEQ ID NO 2), BII1F2, BII5 (SEQ ID NO 3) and BII5F2, respectively. The dinitrophenyl protecting group on histidine was removed using a 20-fold molar excess of thiophenol. Peptides were cleaved from the resin by treatment with HF/anisole (90:10) at 0° C. for 2 h and then precipitated with cold Et₂O. Crude peptides were purified by RP-HPLC [Vydac C₁₈, 10 μM, 10 mm×250 mm]. The purities of peptides were more than 95% as judged by analytical RP-HPLC [Vydac C₁₈, 5 μM, 4 mm×250 mm]. The molar masses of peptides were determined MALDI-TOF MS. Peptide concentrations were determined by quantitative amino acid analysis.

### MALDI-TOF MS Characterization:

M2: m/z calcd (M) 2476.4, obsd 2496.1 (M+Na⁺). M2F2: m/z calcd (M) 2692.3, obsd 2693.6 (M+H⁺). M2F5: m/z calcd (M) 3114.2, obsd 3115.5 (M+H⁺). BII1: m/z calcd (M) 2432.4, obsd 2434.9 (M+H⁺). BII1F2: m/z calcd (M) 2649.3, obsd 2650.7 (M+H⁺). BII5: m/z calcd (M) 2002.2, obsd 2003.5 (M+H⁺). BII5F2: m/z calcd (M) 2218.1, obsd 2218.9 (M+H⁺). GLP-1 m/z calcd (M) 3295.6, obsd 3297.6 (M+H⁺); F8 m/z calcd (M) 3445.7, obsd 3447.3 (M+H⁺); F9 m/z calcd (M) 3389.7, obsd 3398.8 (M+H⁺); F89 m/z calcd (M) 3537.7, obsd 3540.0 (M+H⁺); F10 m/z calcd (M) 2476.4, obsd 2496.1 (M+Na⁺); F28 m/z calcd (M) 2692.3, obsd 2693.6 (M+H⁺); F29 m/z calcd (M) 3114.2, obsd 3115.5 (M+H⁺); F32 m/z calcd (M) 3114.2, obsd 3115.5 (M+H⁺).

### Example 15

### Antimicrobial Activity

Minimal Inhibitory Concentrations (MIC) were measured against Gram-negative Escherichia coli (ATCC 23716) and Gram-positive Bacillus subtilis (SMY) using mid-logarithmic phase cells. Bacteria from a single colony were grown overnight in Luria broth at 37° C. with agitation. An aliquot was taken and diluted (1:50) in fresh broth and cultured for ˜2 h. The cells (OD₅₉₀=0.5) were diluted to a concentration of 5×10⁵ colony forming units/mL (CFU/mL) for M2, M2F2 and M2F5 or a concentration of 5×10⁴ CFU/mL for BII series peptides. The colony forming units per mL were quantitated by spreading 10-fold serially diluted cell suspensions onto Agar plates in triplicate. Two-fold serial dilution of peptide solutions was performed in a sterile 96-well plate (MICROTEST™) in duplicate to a final volume of 50 μL in each well, followed by addition of 50 μL cell suspension. The plate was incubated at 37° C. for 6 h. The absorbance at 590 nm was monitored using a microtiterplate reader (VERSAmax). The MIC was recorded as the concentration of peptide required for the complete inhibition of cell growth (no change in absorbance).

### Example 16

### Hemolysis Assay

Fresh human red blood cells (hRBCs) were centrifuged at 3,500 rpm and washed with PBS buffer until the supernatant was clear. The hRBCs were then resuspended and diluted to a final concentration of 1% (v/v) in PBS and stored at 4° C. Two-fold serial dilution of peptides in PBS in a 96-well plate resulted in a final volume of 20 μL in each well, to which 80 μL hRBCs was added. The plate was incubated at 37° C. for 1 h, followed by centrifugation at 3,500 rpm for 10 min using a SORVALL tabletop centrifuge. An aliquot (50 μL) of supernatant was transferred to a new 96-well plate containing 50 μL H₂O in each well. The absorbance at 415 nm was measured using a plate reader. Wells containing melittin at 100-400 μg/mL served as positive controls, and wells containing only buffer and hRBCs served as negative controls. The percentage hemolysis was calculated using the equation:

$$Percentage hemolysis = 100 \cdot \frac{( {A}_{415 , peptide} - {A}_{415 , buffer} )}{( {A}_{415 , complete hemolysis} - {A}_{415 , buffer} )}$$

where complete hemolysis is defined as the average absorbance of all wells containing 100-400 μg/nL melittin.

### Example 17

### Protease Stability of Peptides

The proteolytic stability of peptides towards trypsin (from bovine pancreas, EC 3.4.21.4) was determined by an analytical RP-HPLC assay. A standard substrate, N-α-Benzoyl-L-arginine ethyl ester (BABE), was used to check enzymatic activity by measuring absorbance at 254 nm. The enzyme concentration (in 1 mM HCl) was determined by absorbance at 280 nm. In a typical trypsinization experiment, 0.25 mM peptide in 200 μL of PBS buffer (pH 7.4, 10 mM PO₄³⁻, 150 mM NaCl) and 1 μg trypsin for M2, M2F2 and M2F5, and 0.5 μg trypsin for BII1, BII5, BII1F2 and BII5F2 (0.19 mM) were used. The amount of enzyme was optimized so that kinetics of proteolytic reactions could be assayed by RP-HPLC (detection at 230 nm). The peptides were incubated with trypsin at 37° C. over a period of 3 h. Aliquots (10 μL) were taken at different reaction times, diluted with 0.2% TFA (440 μL) and stored at −80° C. A C₁₈ analytical column [J. T. Baker C₁₈, 5 μM, 4 mm×250 mm] was used for separation and quantitation of digested products. The remaining full-length peptide concentration was normalized with respect to the initial concentration. Kinetic data after 3 h were fitted using an exponential decay function using Igor Pro 5.03:

A=a+b·e^{-k′t}

Pseudo first order rate constants were then obtained as the fitted value±one standard deviation by fitting data (<initial 20 mins) using the equation:

ln[A]=−kt+ln[A]₀

where A is the normalized concentration of peptides; k is the pseudo first order rate constant; t is the reaction time in mins; and [A]₀ is the initial concentration of peptides. Each fragment cleaved from the full-length peptides was identified by ESI-MS so that cleavage patterns could be established and compared.

### Example 18

### Circular Dichroism

Circular dichroism spectra were recorded at 25° C. on a JASCO J-715 spectropolarimeter fitted with a PTC-423S single position Peltier temperature controller using a 1 cm pathlength cuvette. TFE titrations were carried out in PBS buffer by changing the percentage of TFE while keeping the concentration of peptides constant (10 μM). Four scans were acquired per sample and averaged to improve the S/N ratio. A baseline was recorded and subtracted after each spectrum. Mean residue ellipticities ([θ], deg·cm²·dmol⁻¹) were calculated using the equation:

[θ]=θ_{obs}×MRW/10·l·c

where θ_{obs} is the measured signal (ellipticity) in millidegrees, l is the optical pathlength of the cell in cm, c is the concentration of the peptide in mg/mL and MRW is the mean residue molecular weight (molecular weight of the peptide divided by the number of residues).

For the GLP-1 studies, spectra were recorded at 5° C. on a JASCO J-715 spectropolarimeter fitted with a PTC-423S single position Peltier temperature controller using a 1 mm pathlength cuvette. Peptides were dissolved in 20 mM sodium phosphate, 20 mM sodium phosphate containing 35% TFE, or 40 mM dodecylphosphate choline at pH 7.4 to deliver a final concentration of 10 μM. Four scans were acquired per sample and averaged to improve the S/N ratio at 20 nm/min scanning speed. A baseline was recorded and subtracted for each spectrum. Mean residue ellipticities ([θ], deg·cm²·dmol⁻¹) were calculated using the equation:

[θ]=θ_{obs}10·l·c·n

where θ_{obs} is the measured signal (ellipticity) in millidegrees, l the optical pathlength of the cell in cm, c the peptide concentration in mol/L and n is the number of residues in protein.

### Example 19

### Analytical Ultracentrifugation

Sedimentation equilibrium experiments were performed for M2, M2F2 and M2F5 at 25° C. on a Beckman XL-I ultracentrifuge. Peptides dissolved in PBS were loaded into equilibrium cells at three different concentrations (25, 50, 100 μM for M2 and M2F5; 50, 100, 200 μM for M2F5). Absorbance data at 230 nm were acquired at three different rotor speeds (35,000, 40,000 and 45,000 rpm) after equilibration for 18 hrs. Data obtained were fitted using the following equation that describes the sedimentation of a single ideal species using Igor Pro 5.03:

Abs=A′ exp(H×M[x²−x₀²])+B

where Abs=absorbance at radius x, A′=absorbance at reference radius x₀, H=(1− V̅ρ)ω²/2RT, V̅=partial specific volume (0.7673 mL/g), ρ=density of solvent (1.0017 g/mL), ω=angular velocity in radians/second, R=gas constant (83,144,000 g/mol·K), T=absolute temperature (298 K), M=apparent molecular weight (Da), and B=solvent absorbance (blank). The partial specific volume of peptides was estimated according to the amino acid composition using the program SEDNTERP.

### Example 20

### X-Ray Crystallography

A crystal of 5,5,5,5′,5′,5′-2S-hexafluoroleucine was grown in MeOH and data were collected at 86 (2) K using a Bruker/Siemens SMART APEX instrument (Mo Kα radiation, λ=0.71073 Å) equipped with a Cryocool NeverIce low temperature device. Data were measured using omega scans of 0.3° per frame for 20 seconds, and a full sphere of data was collected. The structure was solved by direct methods and refined by least squares method on F² using the SHELXTL program package.

### Example 21

### Cell Culture and Receptor Transfection

COS-7 cells were cultured in DME supplemented with 10% FBS, penicillin G sodium (100 units/ml) and streptomycin sulfate (100 μg/ml), 26 mM sodium bicarbonate, pH 7.2 at 37° C., 5% CO₂, and highly humidified atmosphere. COS-7 cells (0.8×10⁶ cells) were plated in 10-cm dish a day before transfection. Cells were transiently transfected using the diethylaminoethyl-dextran (DEAE-Dextran) method, with 5 μg of pcDNA1 vector containing the full-length cDNA encoding the wild type human GLP-1 receptor (hGLP1-R) (kindly provided by Dr. Beinborn Martin, Tufts-New England Medical Center, MA). This genetic construct has been sequenced and confirmed the identity.

### Example 22

### Receptor Binding Assay

COS-7 cells (10 k cells/well) were subcultured onto 24-well tissue culture plates (Falcon, Primaria®, BD sciences, CA) a day after transfection. The next day, competition-binding experiments were carried out at 25° C. for 100 min using 17 pM [¹²⁵I]-exendin (9-39) amide as radioligand. The tested peptides had a final concentration ranging from 3×10⁻⁶ to 3×10⁻¹¹ M in 270 μL buffer. Non-specific binding was determined in the presence of 1 μM unlabeled peptides. Fresh binding buffer was prepared in Hanks' balanced salt solution, containing 0.2% BSA, 0.15 mM phenylmethylsulfonyl fluoride (PMSF), 25 mM HEPES, pH 7.3. Cell monolayers were carefully washed one time before and three times after the incubation with 1 mL binding buffer. Cells were hydrolyzed in 1 N NaOH, washed by 1 N HCl, and transferred to polypropylene tubes (Sigma) for gamma counting using a Beckman Gamma counter 5500B.

### Example 23

Measurement of cAMP Formation

COS-7 cells (100 k cells/well) were passaged onto 24-well plates a day after transfection and cultured for another 24 h. Cells were stimulated with GLP-1 and analogs at 25° C. for 1 h in Dulbecco's modified eagle's medium (without phenol red) supplemented with 1% bovine serum albumin, 1 mM isobutyl-methylxanthine (IBMX), 0.4 μM Pro-Boro-Pro, and 25 mM HEPES, pH 7.4. Pro-Boro-Pro ([1-(2-pyrrolidinylcarbonyl)-2-pyrrolidinyl]boronic acid), a potent DPP IV inhibitor, was kindly provided by Dr. W. W. Bachovchin (Tufts University, MA). The final concentrations of tested peptides were 10-fold increased from 1×10⁻⁶ to 1×10⁻¹¹ M in 270 μL buffer. Upon removal of incubation buffer, the cells were lysed by freeze-thaw method in liquid nitrogen (80 s), followed by addition of 200 μL M-Per to ensure the total lysis of cells. The cAMP was acetylated using acetic anhydride/DIEA and its concentration were determined by competitive binding with [¹²⁵I]-cAMP using a FlashPlate® kit (PerkinElmer Life Sciences). Plate-bound radioactivity was measured using a Packard Topcount® proximity scintillation counter.

### Example 24

### Degradation of Peptides Against DPP IV

The proteolytic stability of peptides towards DPP IV (from porcine kidney, EC 3.4.14.5) was determined by analytical RP-HPLC assay (detection at 230 nm). A chromogenic substrate, Gly-Pro-p-nitroanilide, was employed to calibrate specific activity by measuring absorbance at 410 nm using Δε=8800 M⁻¹·cm⁻¹ in 100 mM Tris-HCl, pH 8.0. At enzyme concentration of 20 unit/L, the peptides (8.3 μM) were separately incubated with DPP IV in 50 mM Tris-HCl, 1 mM EDTA, pH 7.6 at 37° C. over 1 h. Reactions were quenched with 600 μL of 0.2% TFA at time intervals and stored on dry ice until the analysis. An analytical C₁₈ column [J. T. Baker C₁₈, 5 μm, 4 mm×250 mm] was used for separation and quantitation of intact and digested peptides with a binary solvent system can/H₂O/0.1% TFA. First order rate constants were obtained as the fitted value±one standard deviation by fitting with the equation:

ln[A]=−kt+ln[A]₀

where A is the concentration of peptides; k the first order rate constant; t the reaction time in min; and [A]₀ the initial concentration of peptides. The fragments derived from the full-length peptides were manually collected and identified by ESI-MS.

### Example 25

### Data Analysis

Radioligand competition binding and cAMP production concentration-response curves were fitted using GraphPad Prism software version 3.0 (GraphPad, San Diego, Calif.). Normalizations were relative to wt GLP-1 for both binding assays and cAMP assays. IC₅₀ and EC₅₀ values were fitted using nonlinear regression with build-in single-site competition model or sigmoidal model. Data are reported as mean±s.e.m.

### Example 26

Calculation of the Free Energy of Unfolding (ΔG°_{unfolding})

Peptides H and F were designed to form parallel dimeric coiled coils. These peptides have an identical sequence except that all seven of the core leucine (L) residues in H are replaced by 5,5,5,5′,5′-S-hexafluoroleucine (X) in F:

H: CGGAQLKKELQALKKENAQLKWELQALKKELAQ
F: CGGAQXKKEXQAXKKENAQXKWEXQAXKKEXAQ

Accordingly the fluorinated peptide FF contains seven hexafluoroleucine residues per helix.

The free energy of unfolding for a non-fluorinated peptide HH was determined by assuming a two state equilibrium between folded and unfolded states.

F_{HH} [symbol] U_{HH}

Where F_{HH} is the folded species and U_{HH} represents the fully unfolded HH. Data were obtained by monitoring [θ]₂₂₂ as a function of Gdn.HCl concentration. Data were analyzed by the linear extrapolation method to yield the free energy of unfolding. The equilibrium constant and therefore ΔG are easily determined from the average fraction of unfolding. Assuming that the linear dependence of ΔG° with denaturant concentration in the transition region continues to zero concentration, the data can be extrapolated to obtain ΔGH°_{H2O}, the free energy difference in the absence of denaturant.

Previously reported sedimentation equilibrium experiments suggest FF is a tetramer (dimer of the disulfide bonded dimer) in the 2-15 μM concentration range. Therefore, an unfolded monomer-folded dimer equilibrium can be used to calculate ΔG° of unfolding:

F_{FF} [symbol] 2U_{FF}

where K_{d}=[U_{FF}]²/[F_{FF}] (U_{FF}=unfolded FF and F_{FF}=folded dimer of FF with 4 helices). Since the total peptide concentration Po can be given by Pₜ=2][F_{FF}]+[U_{FF}], the observed

√

CD signal Y_{obs} can be described in terms of folded and unfolded baselines, Y_{folded} and Y_{unfolded}, respectively, by the following expression:

$${Y}_{obs} = ( {Y}_{unfolded} - {Y}_{folded} ) \frac{\sqrt{{K}_{d}^{2} + 8 {K}_{d} {P}_{t}} - {K}_{d}}{4 {P}_{t}}$$

Additionally, K_{d} can be expressed in terms of the free energy of unfolding.

K_{d}=exp(−ΔG°_{unfolding}/RT)

Assuming that the apparent free energy difference between folded F_{FF} and unfolded U_{FF} states is linearly depended on the Gdn.HCl concentration, ΔG°_{unfolding} can be written as:

ΔG°_{unfolding}=ΔG°_{H2O}−m[Gdn.HCl]

where ΔG°_{H2O} is the free energy difference in the absence of denaturant and m is the dependency of the unfolding transition with respect to the concentration of Gdn.HCl. The data was fit for two parameters, namely ΔG°_{H2O} and m by nonlinear least squared fitting (KaliedaGraph v 3.5).

### INCORPORATION BY REFERENCE

All of the U.S. patents and U.S. patent application publications cited herein are hereby incorporated by reference. Expressly incorporated by reference in its entirety is U.S. patent application Ser. No. 10/468,574, filed Feb. 25, 2002.

### Equivalents

Those skilled in the art will recognize, or be able to ascertain using no more than routine experimentation, many equivalents to the specific embodiments of the invention described herein. Such equivalents are intended to be encompassed by the following claims.

1. A method for preparing a modified peptide, comprising
(a) identifying a natural or non-natural peptide; and
(b) synthesizing a modified peptide based on the sequence of said natural or non-natural peptide;
wherein at least one amino acid of the natural or non-natural peptide is replaced by at least one fluorinated amino acid in said modified polypeptide; and said modified polypeptide has increased stability relative to said natural or non-natural peptide.

2-38. (canceled)

39. The method of claim 1, wherein said at least one fluorinated amino acid is selected from the group consisting of trifluoroleucine, 4,4,4-trifluorovaline, 5,5,5-trifluoroleucine, trifluorovaline, hexafluorovaline, trifluoroisoleucine, trifluoronorvaline, hexafluoroleucine, 5,5,5,5′,5′,5′-hexafluoroleucine, trifluoromethionine, trifluoromethylmethionine and fluorophenylalanine.

40-88. (canceled)

89. The method of claim 1, wherein said natural or non-natural polypeptide has the sequence GIGKFLHAAKKFAKAFVAEIMNS.

90. The method of claim 1, wherein said natural or non-natural polypeptide has the sequence RAGLQFPVGRVHRLLRK.

91. The method of claim 1, wherein said natural or non-natural polypeptide has the sequence TRSSRAGLQFPVGRVHRLLRK.

92. The method of claim 1, wherein said natural or non-natural polypeptide has the sequence QHWSYLLRP.

93. The method of claim 1, wherein said natural or non-natural polypeptide has the sequence KCNTATCATQRLANFLVHSSNNFGPILPPTNVGSNTY.

94. The method of claim 1, wherein said natural or non-natural polypeptide has the sequence HGEGTFTSDLSKQMEEEAVRXIEWLKNGGPSSGAPPPS.

95. The method of claim 1, wherein said natural or non-natural polypeptide has the sequence HAEGTFTSDVSSYLEGQAAKEFIAWLVKGR.

96. The method of claim 1, wherein said natural or non-natural polypeptide has the sequence SPKMVQGSGCFGRKMDRISSSSGLGCKVLRRK.

97. The method of claim 1, wherein said natural or non-natural polypeptide has the sequence YTSLIHSLIEESQNQQELNEQELLELDKWASLWNWF.

98. The method of claim 1, wherein said natural or non-natural polypeptide has the sequence VVYTDCTESGQNLCLCEGSNVCGQGNKClLGSDGEKNQCVTGEGTPKPQSHNDGDFEEI PEEYLQ.

99. The method of claim 1, wherein said natural or non-natural polypeptide has the sequence MPLWVFFFVILTLSNSSHCSPPPPLTLRMRRYADAIFTNSYRKVLGQLSARKLLQDIMSR QQGESNQERGARARLGRQVDSMWAEQKQMELESILVALLQKHSRNSQG.

100. The method of claim 1, wherein said natural or non-natural polypeptide has the sequence MKPIQKLLAGLILLTSCVEGCSSQHWSYGLRPGGKRDAENLIDSFQEIVKEVGQLAETQR FECTTHQPRSPLRDLKGALESLIEEETGQKKI.

101. The method of claim 1, wherein said natural or non-natural polypeptide has the sequence MALWMRLLPLLALLALWGPDPAAAFVNQHLCGSHLVEALYLVCGERGFFYTPKTRRE AEDLQVGQVELGGGPGAGSLQPLALEGSLQKRGIVEQCCTSICSLYQLENYCN.

102. A polypeptide comprising at least one fluorinated amino acid wherein said polypeptide has a sequence selected from the group consisting of GIGKFXHAAKKFAKAFVAEXMNS; GIGKFXHAXKKFXKAFXAEXMNS; RAGLQFPVGRVHRXXRK; TRSSRAGLQFPVGRVHRXXRK; HXEGTFTSDVSSYLEGQAAKEFIAWLVKGR; HAXGTFTSDVSSYLEGQAAKEFIAWLVKGR; HXXGTFTSDVSSYLEGQAAKEFIAWLVKGR; HXXGTFTSDVSSYLEGQAAKEFIAWLVKGR; HXEGTFTSDVSSYLEGQAAKEXIAWLVKGR; HAXGTFTSDVSSYLEGQAAKEFXAWLVKGR; and HXEGTFTSDVSSYLEGQAAKEFIAWXVKGR; wherein X is independently a fluorinated amino acid.

103. The polypeptide of claim 102, wherein said polypeptide has the sequence GIGKFXHAAKKFAKAFVAEXMNS.

104. The polypeptide of claim 102, wherein said polypeptide has the sequence GIGKFXHAXKKFXKAFXAEXMNS.

105. The polypeptide of claim 102, wherein said polypeptide has the sequence RAGLQFPVGRVHRXXRK.

106. The polypeptide of claim 102, wherein said polypeptide has the sequence TRSSRAGLQFPVGRVHRXXRK.

107. The polypeptide of claim 102, wherein said polypeptide has a sequence selected from the group consisting of HXEGTFTSDVSSYLEGQAAKEFIAWLVKGR; HAXGTFTSDVSSYLEGQAAKEFIAWLVKGR; HXXGTFTSDVSSYLEGQAAKEFIAWLVKGR; HXXGTFTSDVSSYLEGQAAKEFIAWLVKGR; HXEGTFTSDVSSYLEGQAAKEXIAWLVKGR; HAXGTFTSDVSSYLEGQAAKEFXAWLVKGR; and HXEGTFTSDVSSYLEGQAAKEFIAWXVKGR.

108. The polypeptide of claim 102, wherein said polypeptide has the sequence HXEGTFTSDVSSYLEGQAAKEFIAWLVKGR.

109. The polypeptide of claim 102, wherein said polypeptide has the sequence HAXGTFTSDVSSYLEGQAAKEFIAWLVKGR.

110. The polypeptide of claim 102, wherein said polypeptide has the sequence HXXGTFTSDVSSYLEGQAAKEFIAWLVKGR.

111. The polypeptide of claim 102, wherein said polypeptide has the sequence HXXGTFTSDVSSYLEGQAAKEFIAWLVKGR.

112. The polypeptide of claim 102, wherein said polypeptide has the sequence HXEGTFTSDVSSYLEGQAAKEXIAWLVKGR.

113. The polypeptide of claim 102, wherein said polypeptide has the sequence HAXGTFTSDVSSYLEGQAAKEFXAWLVKGR.

114. The polypeptide of claim 102, wherein said polypeptide has the sequence HXEGTFTSDVSSYLEGQAAKEFIAWXVKGR.

115. The polypeptide of claim 102, wherein the fluorinated amino acid X is independently selected from the group consisting of trifluoroleucine, 4,4,4-trifluorovaline, 5,5,5-trifluoroleucine, trifluorovaline, hexafluorovaline, trifluoroisoleucine, trifluoronorvaline, hexafluoroleucine, 5,5,5,5′,5′,5′-hexafluoroleucine, trifluoromethionine, trifluoromethylmethionine and fluorophenylalanine.

116. A polypeptide, comprising at least one fluorinated amino acid replacement for at least one replaced natural amino acid, wherein said at least one fluorinated amino acid replacement is selected from the group consisting of trifluoroleucine, 4,4,4-trifluorovaline, 5,5,5-trifluoroleucine, trifluorovaline, hexafluorovaline, trifluoroisoleucine, trifluoronorvaline, hexafluoroleucine, 5,5,5,5′,5′,5′-hexafluoroleucine, trifluoromethionine, trifluoromethylmethionine and fluorophenylalanine; and said polypeptide is selected from the group consisting of: GIGKFLHAAKKFAKAFVAEIMNS, RAGLQFPVGRVHRLLRK, TRSSRAGLQFPVGRVHRLLRK, QHWSYLLRP, KCNTATCATQRLANFLVHSSNNFGPILPPTNVGSNTY, HGEGTFTSDLSKQMEEEAVRXIEWLKNGGPSSGAPPPS, HAEGTFTSDVSSYLEGQAAKEFIAWLVKGR, SPKMVQGSGCFGRKMDRISSSSGLGCKVLRRK, YTSLIHSLIEESQNQQELNEQELLELDKWASLWNWF, VVYTDCTESGQNLCLCEGSNVCGQGNKClLGSDGEKNQCVTGEGTPKPQSHNDGDFEEI PEEYLQ, MPLWVFFFVILTLSNSSHCSPPPPLTLRMRRYADAIFTNSYRKVLGQLSARKLLQDIMSR QQGESNQERGARARLGRQVDSMWAEQKQMELESILVALLQKHSRNSQ, MKPIQKLLAGLILLTSCVEGCSSQHWSYGLRPGGKRDAENLIDSFQEIVKEVGQLAETQR FECTTHQPRSPLRDLKGALESLIEEETGQKKI, and MALWMRLLPLLALLALWGPDPAAAFVNQHLCGSHLVEALYLVCGERGFFYTPKTRRE AEDLQVGQVELGGGPGAGSLQPLALEGSLQKRGIVEQCCTSICSLYQLENYCN.

117-121. (canceled)

122. A polypeptide, comprising at least one fluorinated amino acid replacement, wherein said at least one fluorinated amino acid replacement is selected from the group consisting of trifluoroleucine, 5,5,5-trifluoroleucine, hexafluoroleucine, and 5,5,5,5′,5′,5′-hexafluoroleucine; each instance of X is independently leucine or a fluorinated amino acid replacement; and said polypeptide is selected from the group consisting of: GIGKFXHAAKKFAKAFVAEIMNS, RAGXQFPVGRVHRXXRK, TRSSRAGXQFPVGRVHRXXRK, QHWSYXXRP, KCNTATCATQRXANFXVHSSNNFGPIXPPTNVGSNTY, HGEGTFTSDXSKQMEEEAVRXIEWXKNGGPSSGAPPPS, HAEGTFTSDVSSYXEGQAAKEFIAWXVKGR, SPKMVQGSGCFGRKMDRISSSSGXGCKVXRRK, YTSXIHSXIEESQNQQEXNEQEXXEXDKWASXWNWF, VVYTDCTESGQNXCXCEGSNVCGQGNKCIXGSDGEKNQCVTGEGTPKPQSHNDGDFEE IPEEYXQ, MPXWVFFFVIXTXSNSSHCSPPPPXTXRMRRYADAIFTNSYRKVXGQXSARKXXQDIMS RQQGESNQERGARARXGRQVDSMWAEQKQMEXESIXVAXXQKHSRNSQG, MKPIQKXXAGXIXXTSCVEGCSSQHWSYGXRPGGKRDAENXIDSFQEIVKEVGQXAET QRFECTTHQPRSPXRDXKGAXESXIEEETGQKKI, and MAXWMRXXPXXAXXAXWGPDPAAAFVNQHXCGSHXVEAXYXVCGERGFFYTPKTRR EAEDXQVGQVEXGGGPGAGSXQPXAXEGSXQKRGIVEQCCTSICSXYQXENYCN.

123. The polypeptide of claim 122, wherein said at least one fluorinated amino acid replacement is selected from the group consisting of 5,5,5,5′,5′,5′-hexafluoroleucine.

124. A polypeptide, comprising at least one fluorinated amino acid replacement for at least one replaced natural amino acid, wherein said at least one fluorinated amino acid replacement is selected from the group consisting of trifluoroleucine, 4,4,4-trifluorovaline, 5,5,5-trifluoroleucine, trifluorovaline, hexafluorovaline, trifluoroisoleucine, trifluoronorvaline, hexafluoroleucine, 5,5,5,5′,5′,5′-hexafluoroleucine, trifluoromethionine, trifluoromethylmethionine and fluorophenylalanine; each instance of X is independently a fluorinated amino acid replacement; and said polypeptide is selected from the group consisting of HXEGTFTSDVSSYLEGQAAKEFIAWLVKGR; HAXGTFTSDVSSYLEGQAAKEFIAWLVKGR; HXXGTFTSDVSSYLEGQAAKEFIAWLVKGR; HXXGTFTSDVSSYLEGQAAKEFIAWLVKGR; HXEGTFTSDVSSYLEGQAAKEXIAWLVKGR; HAXGTFTSDVSSYLEGQAAKEFXAWLVKGR; and HXEGTFTSDVSSYLEGQAAKEFIAWXVKGR.

125-129. (canceled)

130. A polypeptide, comprising at least one fluorinated amino acid replacement, wherein said at least one fluorinated amino acid replacement is selected from the group consisting of trifluoroleucine, 5,5,5-trifluoroleucine, hexafluoroleucine, and 5,5,5,5′,5′,5′-hexafluoroleucine; each instance of X is independently leucine or a fluorinated amino acid replacement; and said polypeptide is selected from the group consisting of HXEGTFTSDVSSYLEGQAAKEFIAWLVKGR; HAXGTFTSDVSSYLEGQAAKEFIAWLVKGR; HXXGTFTSDVSSYLEGQAAKEFIAWLVKGR; HXXGTFTSDVSSYLEGQAAKEFIAWLVKGR; HXEGTFTSDVSSYLEGQAAKEXIAWLVKGR; HAXGTFTSDVSSYLEGQAAKEFXAWLVKGR; and HXEGTFTSDVSSYLEGQAAKEFIAWXVKGR.

131. The polypeptide of claim 130, wherein said at least one fluorinated amino acid replacement is selected from the group consisting of 5,5,5,5′,5′,5′-hexafluoroleucine.

132. A polypeptide comprising at least one radiolabeled amino acid wherein said polypeptide has the sequence DLSK*QMEEEAVRLFIEWLKNGGPSSGAPPPS; wherein K* is a radiolabeled amino acid.
